(19) United States
(12) Patent Application Publication
(10) Pub. No.: US 2020/0399672 A1
(43) Pub. Date: Dec. 24, 2020

(54) IMMUNOCHROMATOGRAPHIC ANALYSIS DEVICE FOR CHIKUNGUNYA VIRUS DETECTION

(71) Applicants: OSAKA UNIVERSITY, Suita-shi, Osaka (JP); TANAKA KIKINZOKU KOGYO K.K., Tokyo (JP)

(72) Inventors: Tatsuo SHIODA, Osaka (JP); Emi NAKAYAMA, Osaka (JP); Keita SUZUKI, Kanagawa (JP); Hisahiko IWAMOTO, Kanagawa (JP)

(21) Appl. No.: 16/968,467

(22) PCT Filed: Feb. 8, 2019
(86) PCT No.: PCT/JP2019/004755

§ 371 Date: Aug. 7, 2020

(30) Foreign Application Priority Data
Feb. 9, 2018 (JP) 2018-022527

(51) Int. Cl. C12Q 1/04; G01N 33/569; G01N 33/543

(52) U.S. Cl. CPC C12Q 1/04; G01N 33/543; G01N 33/569

(57) ABSTRACT

The present invention relates to an immunochromatographic analysis device for detecting chikungunya virus, including a sample loading part, a labeling substance retaining part, a chromatographic medium part having a detection part, and an absorption part, wherein the labeling substance retaining part contains a first antibody against the chikungunya virus, including a specific heavy chain variable region and a specific light chain variable region or an antigen-binding fragment thereof, and the detection part contains a second antibody against the chikungunya virus, including a specific heavy chain variable region and the light chain variable region or an antigen-binding fragment thereof, and a third antibody against the chikungunya virus, including a specific heavy chain variable region and the light chain variable region or an antigen-binding fragment thereof.

### TECHNICAL FIELD

The present invention relates to an immunochromatographic analysis device, an immunochromatographic analysis kit, and an immunochromatographic analysis method for detecting chikungunya virus.

### BACKGROUND ART

Chikungunya virus (CHIKV) proliferates in arthropods such as mosquitoes and mites and spreads to vertebrates by their blood-sucking activity. Infection with chikungunya virus causes high fever called chikungunya fever and severe joint pain. Sometimes serious symptoms may be caused, and establishment of a rapid diagnostic method for early treatment has been demanded.

Chikungunya virus is classified in the genus Alphavirus of the family Togaviridae, and is a spherical RNA virus. It is known that when the genotypes are broadly classified, there are three genotypes: ECSA genotype, WA genotype, and Asian genotype. In all the genotypes, the ⅓ region on the 3′ end side of genomic RNA encodes five structural proteins of CP, E3, E2, 6K, and E1, and the ⅔ region on the 5′ end side encodes four nonstructural proteins of nsP1, nsP2, nsP3, and nsP4.

Non-Patent Literatures 1 and 2 disclose methods for detecting ECSA genotype and Asian genotype chikungunya viruses by immunochromatography using a mouse monoclonal antibody that recognizes chikungunya virus E1 protein. Non-Patent Literature 3 discloses CK47 which is a mouse monoclonal antibody that reacts with chikungunya virus E1 protein.

Further, Patent Literature 1 discloses a method in which an immune complex is formed using an anti-chikungunya virus E2 antibody which is an antibody against chikungunya virus E2 protein, and the presence or absence of chikungunya virus is detected based on the presence or absence of the immune complex.

### CITATION LIST

### Patent Literature

Patent Literature 1: JP-T-2010-538291

### Non-Patent Literature

Non-Patent Literature 1: Journal of Clinical Microbiology, February 2015 Volume 53 Number 2
Non-Patent Literature 2: Clinical Microbiology and Infection 24 (2018) 78e81
Non-Patent Literature 3: Virology, 464-465 (2014) 111-117

### SUMMARY OF INVENTION

### Technical Problem

However, the antibody used in the immunochromatography described in Non-Patent Literature 2 has good sensitivity to ECSA genotype chikungunya virus, but has low sensitivity to Asian genotype chikungunya virus.

In addition, as disclosed in Non-Patent Literature 3, the antibody used in the immunochromatography described in Non-Patent Literature 1 has a problem that the activity is significantly lowered by a point mutation in the virus protein.

Further, Patent Literature 1 discloses an antibody against chikungunya virus E2 protein, but no particular attention is paid to whether the antibody has reactivity with various genotypes of chikungunya virus, and the reactivity with the respective genotypes and sensitivity thereto are unknown. In addition, it is known that a host (human) produces a neutralizing antibody against chikungunya virus E2 protein, and when the chikungunya virus E2 protein is determined as a target of an immunological measurement system, it may be affected by competitive inhibition of an antibody derived from the host.

The present invention has been made in view of the above-mentioned technical problems, and an object of the present invention is to achieve detection promptly and with high sensitivity even between chikungunya viruses with different genotypes. In addition, an object of the present invention is to detect even chikungunya virus in which a part of the structural protein is mutated promptly and with high sensitivity. Further, an object of the present invention is to specifically detect chikungunya virus by suppressing cross-reaction with another virus (for example, Sindbis virus, SINV) other than the chikungunya virus.

### Solution to Problem

As a result of intensive studies to achieve the above objects, the present inventors found that the objects can be achieved by using specific antibodies against chikungunya virus in combination in a labeling substance retaining part and a detection part in an immunochromatographic analysis device, and thus completed the present invention.

Therefore, the present invention is as follows.

1. An immunochromatographic analysis device for detecting chikungunya virus, including a sample loading part, a labeling substance retaining part, a chromatographic medium part having a detection part, and an absorption part, wherein

the labeling substance retaining part contains the following antibody (A) or an antigen-binding fragment thereof, and

the detection part contains the following antibody (B) or an antigen-binding fragment thereof, and the following antibody (C) or an antigen-binding fragment thereof,

(A) an antibody against the chikungunya virus, including the following heavy chain variable region (1) and the following light chain variable region (2),

(1) a heavy chain variable region including a heavy chain complementarity determining region (CDRH) 1, CDRH2, and CDRH3,

CDRH1 being a polypeptide containing the following amino acid sequence (H1-A),

CDRH2 being a polypeptide containing the following amino acid sequence (H2-A), and

CDRH3 being a polypeptide containing the following amino acid sequence (H3-A),

(H1-A) the following amino acid sequence (H1-A1), (H1-A2), or (H1-A3),
(H1-A1) an amino acid sequence of SEQ ID NO: 1,
(H1-A2) an amino acid sequence having 80% or more identity with the amino acid sequence of SEQ ID NO: 1,
(H1-A3) an amino acid sequence in which one or several amino acids are deleted, substituted, inserted, and/or added in the amino acid sequence of SEQ ID NO: 1,

(H2-A) the following amino acid sequence (H2-A1), (H2-A2), or (H2-A3),
(H2-A1) an amino acid sequence of SEQ ID NO: 2,
(H2-A2) an amino acid sequence having 80% or more identity with the amino acid sequence of SEQ ID NO: 2,
(H2-A3) an amino acid sequence in which one or several amino acids are deleted, substituted, inserted, and/or added in the amino acid sequence of SEQ ID NO: 2,

(H3-A) the following amino acid sequence (H3-A1), (H3-A2), or (H3-A3),
(H3-A1) an amino acid sequence of SEQ ID NO: 3,
(H3-A2) an amino acid sequence having 80% or more identity with the amino acid sequence of SEQ ID NO: 3,
(H3-A3) an amino acid sequence in which one or several amino acids are deleted, substituted, inserted, and/or added in the amino acid sequence of SEQ ID NO: 3,

(2) a light chain variable region including a light chain complementarity determining region (CDRL) 1, CDRL2, and CDRL3,

CDRL1 being a polypeptide containing the following amino acid sequence (L1-A),

CDRL2 being a polypeptide containing the following amino acid sequence (L2-A), and

CDRL3 being a polypeptide containing the following amino acid sequence (L3-A),

(L1-A) the following amino acid sequence (L1-A1), (L1-A2), or (L1-A3),
(L1-A1) an amino acid sequence of SEQ ID NO: 4,
(L1-A2) an amino acid sequence having 80% or more identity with the amino acid sequence of SEQ ID NO: 4,
(L1-A3) an amino acid sequence in which one or several amino acids are deleted, substituted, inserted, and/or added in the amino acid sequence of SEQ ID NO: 4,

(L2-A) the following amino acid sequence (L2-A1), (L2-A2), or (L2-A3),
(L2-A1) an amino acid sequence of SEQ ID NO: 5,
(L2-A2) an amino acid sequence having 80% or more identity with the amino acid sequence of SEQ ID NO: 5,
(L2-A3) an amino acid sequence in which one or several amino acids are deleted, substituted, inserted, and/or added in the amino acid sequence of SEQ ID NO: 5,

(L3-A) the following amino acid sequence (L3-A1), (L3-A2), or (L3-A3),
(L3-A1) an amino acid sequence of SEQ ID NO: 6,
(L3-A2) an amino acid sequence having 80% or more identity with the amino acid sequence of SEQ ID NO: 6,
(L3-A3) an amino acid sequence in which one or several amino acids are deleted, substituted, inserted, and/or added in the amino acid sequence of SEQ ID NO: 6,

(B) an antibody against the chikungunya virus, including the following heavy chain variable region (3) and the light chain variable region (2),

(3) a heavy chain variable region including a heavy chain complementarity determining region (CDRH) 1, CDRH2, and CDRH3,

CDRH1 being a polypeptide containing the following amino acid sequence (H1-B),

CDRH2 being a polypeptide containing the following amino acid sequence (H2-B), and

CDRH3 being a polypeptide containing the following amino acid sequence (H3-B),

(H1-B) the following amino acid sequence (H1-B1), (H1-B2), or (H1-B3),
(H1-B1) an amino acid sequence of SEQ ID NO: 7,
(H1-B2) an amino acid sequence having 80% or more identity with the amino acid sequence of SEQ ID NO: 7,
(H1-B3) an amino acid sequence in which one or several amino acids are deleted, substituted, inserted, and/or added in the amino acid sequence of SEQ ID NO: 7,

(H2-B) the following amino acid sequence (H2-B1), (H2-B2), or (H2-B3),
(H2-B1) an amino acid sequence of SEQ ID NO: 8,
(H2-B2) an amino acid sequence having 80% or more identity with the amino acid sequence of SEQ ID NO: 8,
(H2-B3) an amino acid sequence in which one or several amino acids are deleted, substituted, inserted, and/or added in the amino acid sequence of SEQ ID NO: 8,

(H3-B) the following amino acid sequence (H3-B1), (H3-B2), or (H3-B3),
(H3-B1) an amino acid sequence of SEQ ID NO: 9,
(H3-B2) an amino acid sequence having 80% or more identity with the amino acid sequence of SEQ ID NO: 9,
(H3-B3) an amino acid sequence in which one or several amino acids are deleted, substituted, inserted, and/or added in the amino acid sequence of SEQ ID NO: 9, and

(C) an antibody against the chikungunya virus, including the following heavy chain variable region (4) and the light chain variable region (2),

(4) a heavy chain variable region including CDRH1, CDRH2, and CDRH3,

CDRH1 being a polypeptide containing the following amino acid sequence (H1-C),

CDRH2 being a polypeptide containing the following amino acid sequence (H2-C), and

CDRH3 being a polypeptide containing the following amino acid sequence (H3-C),

(H1-C) the following amino acid sequence (H1-C1), (H1-C2), or (H1-C3),
(H1-C1) an amino acid sequence of SEQ ID NO: 10,
(H1-C2) an amino acid sequence having 80% or more identity with the amino acid sequence of SEQ ID NO: 10,
(H1-C3) an amino acid sequence in which one or several amino acids are deleted, substituted, inserted, and/or added in the amino acid sequence of SEQ ID NO: 10,

(H2-C) the following amino acid sequence (H2-C1), (H2-C2), or (H2-C3),
(H2-C1) an amino acid sequence of SEQ ID NO: 11,
(H2-C2) an amino acid sequence having 80⁰ or more identity with the amino acid sequence of SEQ ID NO: 11,
(H2-C3) an amino acid sequence in which one or several amino acids are deleted, substituted, inserted, and/or added in the amino acid sequence of SEQ ID NO: 11,

(H3-C) the following amino acid sequence (H3-C1), (H3-C2), or (H3-C3),
(H3-C1) an amino acid sequence of SEQ ID NO: 12,
(H3-C2) an amino acid sequence having 80% or more identity with the amino acid sequence of SEQ ID NO: 12,
(H3-C3) an amino acid sequence in which one or several amino acids are deleted, substituted, inserted, and/or added in the amino acid sequence of SEQ ID NO: 12.

2. An immunochromatographic analysis device for detecting chikungunya virus, including a sample loading part, a labeling substance retaining part, a chromatographic medium part having a detection part, and an absorption part, wherein

the labeling substance retaining part contains the following antibody (A) or an antigen-binding fragment thereof, and

the detection part contains the following antibody (B) or an antigen-binding fragment thereof, and the following antibody (C) or an antigen-binding fragment thereof,

(A) an antibody against the chikungunya virus, including the following heavy chain variable region (1) and the following light chain variable region (2),

(1) a heavy chain variable region including a polypeptide composed of the following amino acid sequence (H-A),

(H-A) the following amino acid sequence (H-A1), (H-A2), or (H-A3),
(H-A1) an amino acid sequence of SEQ ID NO: 13,
(H-A2) an amino acid sequence having 80% or more identity with the amino acid sequence of SEQ ID NO: 13,
(H-A3) an amino acid sequence in which one or several amino acids are deleted, substituted, inserted, and/or added in the amino acid sequence of SEQ ID NO: 13,

(2) a light chain variable region including a polypeptide composed of the following amino acid sequence (L-A),

(L-A) the following amino acid sequence (L-A1), (L-A2), or (L-A3),
(L-A1) an amino acid sequence of SEQ ID NO: 14,
(L-A2) an amino acid sequence having 80% or more identity with the amino acid sequence of SEQ ID NO: 14,
(L-A3) an amino acid sequence in which one or several amino acids are deleted, substituted, inserted, and/or added in the amino acid sequence of SEQ ID NO: 14,

(B) an antibody against the chikungunya virus, including the following heavy chain variable region (3) and the light chain variable region (2),

(3) a heavy chain variable region including a polypeptide composed of the following amino acid sequence (H-B),

(H-B) the following amino acid sequence (H-B1), (H-B2), or (H-B3),
(H-B1) an amino acid sequence of SEQ ID NO: 15,
(H-B2) an amino acid sequence having 80% or more identity with the amino acid sequence of SEQ ID NO: 15,
(H-B3) an amino acid sequence in which one or several amino acids are deleted, substituted, inserted, and/or added in the amino acid sequence of SEQ ID NO: 15,

(C) an antibody against the chikungunya virus, including the following heavy chain variable region (4) and the light chain variable region (2),

(4) a heavy chain variable region including a polypeptide composed of the following amino acid sequence (H-C),

(H-C) the following amino acid sequence (H-C1), (H-C2), or (H-C3),
(H-C1) an amino acid sequence of SEQ ID NO: 16,
(H-C2) an amino acid sequence having 80% or more identity with the amino acid sequence of SEQ ID NO: 16,
(H-C3) an amino acid sequence in which one or several amino acids are deleted, substituted, inserted, and/or added in the amino acid sequence of SEQ ID NO: 16.

3. An immunochromatographic analysis device for detecting chikungunya virus, including a sample loading part, a labeling substance retaining part, a chromatographic medium part having a detection part, and an absorption part, wherein

the labeling substance retaining part contains the following antibody (A) or an antigen-binding fragment thereof, and

the detection part contains the following antibody (B) or an antigen-binding fragment thereof, and the following antibody (C) or an antigen-binding fragment thereof,

(A) an antibody against the chikungunya virus, including the following heavy chain (1) and the following light chain (2),

(1) a heavy chain including a polypeptide composed of the following amino acid sequence (HA),

(HA) the following amino acid sequence (HA1), (HA2), or (HA3),
(HA1) an amino acid sequence of SEQ ID NO: 17,
(HA2) an amino acid sequence having 80% or more identity with the amino acid sequence of SEQ ID NO: 17,
(HA3) an amino acid sequence in which one or several amino acids are deleted, substituted, inserted, and/or added in the amino acid sequence of SEQ ID NO: 17,

(2) a light chain including a polypeptide composed of the following amino acid sequence (LA),

(LA) the following amino acid sequence (LA1), (LA2), or (LA3),
(LA1) an amino acid sequence of SEQ ID NO: 18,
(LA2) an amino acid sequence having 80% or more identity with the amino acid sequence of SEQ ID NO: 18,
(LA3) an amino acid sequence in which one or several amino acids are deleted, substituted, inserted, and/or added in the amino acid sequence of SEQ ID NO: 18,

(B) an antibody against the chikungunya virus, including the following heavy chain (3) and the light chain (2),

(3) a heavy chain including a polypeptide composed of the following amino acid sequence (HB),

(HB) the following amino acid sequence (HB1), (HB2), or (HB3),
(HB1) an amino acid sequence of SEQ ID NO: 19,
(HB2) an amino acid sequence having 80% or more identity with the amino acid sequence of SEQ ID NO: 19,
(HB3) an amino acid sequence in which one or several amino acids are deleted, substituted, inserted, and/or added in the amino acid sequence of SEQ ID NO: 19,

(C) an antibody against the chikungunya virus, including the following heavy chain (4) and the light chain (2),

(4) a heavy chain including a polypeptide composed of the following amino acid sequence (HC),

(HC) the following amino acid sequence (HC1), (HC2), or (HC3),
(HC1) an amino acid sequence of SEQ ID NO: 20,
(HC2) an amino acid sequence having 80% or more identity with the amino acid sequence of SEQ ID NO: 20,
(HC3) an amino acid sequence in which one or several amino acids are deleted, substituted, inserted, and/or added in the amino acid sequence of SEQ ID NO: 20.

4. The immunochromatographic analysis device according to any one of the above 1 to 3, for detecting a chikungunya virus envelope glycoprotein of the chikungunya virus.

5. The immunochromatographic analysis device according to the above 4, wherein the chikungunya virus envelope glycoprotein is chikungunya virus E1 protein.

6. The immunochromatographic analysis device according to any one of the above 1 to 5, wherein the antibody (A) or an antigen-binding fragment thereof, the antibody (B) or an antigen-binding fragment thereof, and the antibody (C) or an antigen-binding fragment thereof specifically bind to chikungunya virus having at least any one genotype of ECSA genotype, Asian genotype, and WA genotype.

7. The immunochromatographic analysis device according to any one of the above 1 to 6, wherein

the labeling substance retaining part further contains the following antibody (D) or an antigen-binding fragment thereof, and

the detection part further contains the following antibody (E) or an antigen-binding fragment thereof,

(D) an antibody against the chikungunya virus, including the following heavy chain variable region (1) and the following light chain variable region (2),

(1) a heavy chain variable region including a heavy chain complementarity determining region (CDRH) 1, CDRH2, and CDRH3,

CDRH1 being a polypeptide containing the following amino acid sequence (H1-D),

CDRH2 being a polypeptide containing the following amino acid sequence (H2-D), and

CDRH3 being a polypeptide containing the following amino acid sequence (H3-D),

(H1-D) the following amino acid sequence (H1-D1), (H1-D2), or (H1-D3),
(H1-D1) an amino acid sequence of SEQ ID NO: 21,
(H1-D2) an amino acid sequence having 80% or more identity with the amino acid sequence of SEQ ID NO: 21,
(H1-D3) an amino acid sequence in which one or several amino acids are deleted, substituted, inserted, and/or added in the amino acid sequence of SEQ ID NO: 21,

(H2-D) the following amino acid sequence (H2-D1), (H2-D2), or (H2-D3),
(H2-D1) an amino acid sequence of SEQ ID NO: 22,
(H2-D2) an amino acid sequence having 80% or more identity with the amino acid sequence of SEQ ID NO: 22,
(H2-D3) an amino acid sequence in which one or several amino acids are deleted, substituted, inserted, and/or added in the amino acid sequence of SEQ ID NO: 22,

(H3-D) the following amino acid sequence (H3-D1), (H3-D2), or (H3-D3),
(H3-D1) an amino acid sequence of SEQ ID NO: 23,
(H3-D2) an amino acid sequence having 80% or more identity with the amino acid sequence of SEQ ID NO: 23,
(H3-D3) an amino acid sequence in which one or several amino acids are deleted, substituted, inserted, and/or added in the amino acid sequence of SEQ ID NO: 23,

(2) a light chain variable region including a light chain complementarity determining region (CDRL) 1, CDRL2, and CDRL3,

CDRL1 being a polypeptide containing the following amino acid sequence (L1-A),

CDRL2 being a polypeptide containing the following amino acid sequence (L2-A), and

CDRL3 being a polypeptide containing the following amino acid sequence (L3-A),

(L1-A) the following amino acid sequence (L1-A1), (L1-A2), or (L1-A3),
(L1-A1) an amino acid sequence of SEQ ID NO: 4,
(L1-A2) an amino acid sequence having 80% or more identity with the amino acid sequence of SEQ ID NO: 4,
(L1-A3) an amino acid sequence in which one or several amino acids are deleted, substituted, inserted, and/or added in the amino acid sequence of SEQ ID NO: 4,

(L2-A) the following amino acid sequence (L2-A1), (L2-A2), or (L2-A3),
(L2-A1) an amino acid sequence of SEQ ID NO: 5,
(L2-A2) an amino acid sequence having 80% or more identity with the amino acid sequence of SEQ ID NO: 5,
(L2-A3) an amino acid sequence in which one or several amino acids are deleted, substituted, inserted, and/or added in the amino acid sequence of SEQ ID NO: 5,

(L3-A) the following amino acid sequence (L3-A1), (L3-A2), or (L3-A3),
(L3-A1) an amino acid sequence of SEQ ID NO: 6,
(L3-A2) an amino acid sequence having 80% or more identity with the amino acid sequence of SEQ ID NO: 6,
(L3-A3) an amino acid sequence in which one or several amino acids are deleted, substituted, inserted, and/or added in the amino acid sequence of SEQ ID NO: 6,

(E) an antibody against the chikungunya virus, including the following heavy chain variable region (3) and the light chain variable region (2),

(3) a heavy chain variable region including a heavy chain complementarity determining region (CDRH) 1, CDRH2, and CDRH3,

CDRH1 being a polypeptide containing the following amino acid sequence (H1-E),

CDRH2 being a polypeptide containing the following amino acid sequence (H2-E), and

CDRH3 being a polypeptide containing the following amino acid sequence (H3-E),

(H1-E) the following amino acid sequence (H1-E1), (H1-E2), or (H1-E3),
(H1-E1) an amino acid sequence of SEQ ID NO: 24,
(H1-E2) an amino acid sequence having 80% or more identity with the amino acid sequence of SEQ ID NO: 24,
(H1-E3) an amino acid sequence in which one or several amino acids are deleted, substituted, inserted, and/or added in the amino acid sequence of SEQ ID NO: 24,

(H2-E) the following amino acid sequence (H2-E1), (H2-E2), or (H2-E3),
(H2-E1) an amino acid sequence of SEQ ID NO: 25,
(H2-E2) an amino acid sequence having 80% or more identity with the amino acid sequence of SEQ ID NO: 25,
(H2-E3) an amino acid sequence in which one or several amino acids are deleted, substituted, inserted, and/or added in the amino acid sequence of SEQ ID NO: 25,

(H3-E) the following amino acid sequence (H3-E1), (H3-E2), or (H3-B3),
(H3-E1) an amino acid sequence of SEQ ID NO: 26,
(H3-E2) an amino acid sequence having 80% or more identity with the amino acid sequence of SEQ ID NO: 26,
(H3-E3) an amino acid sequence in which one or several amino acids are deleted, substituted, inserted, and/or added in the amino acid sequence of SEQ ID NO: 26.

8. The immunochromatographic analysis device according to any one of the above 1 to 6, wherein

the labeling substance retaining part further contains the following antibody (D) or an antigen-binding fragment thereof, and

the detection part further contains the following antibody (E) or an antigen-binding fragment thereof,

(D) an antibody against the chikungunya virus, including the following heavy chain variable region (1) and the following light chain variable region (2),

(1) a heavy chain variable region including a polypeptide composed of the following amino acid sequence (H-D),

(H-D) the following amino acid sequence (H-D1), (H-D2), or (H-D3),
(H-D1) an amino acid sequence of SEQ ID NO: 27,
(H-D2) an amino acid sequence having 80% or more identity with the amino acid sequence of SEQ ID NO: 27,
(H-D3) an amino acid sequence in which one or several amino acids are deleted, substituted, inserted, and/or added in the amino acid sequence of SEQ ID NO: 27,

(2) a light chain variable region including a polypeptide composed of the following amino acid sequence (L-A),

(L-A) the following amino acid sequence (L-A1), (L-A2), or (L-A3),
(L-A1) an amino acid sequence of SEQ ID NO: 14,
(L-A2) an amino acid sequence having 80% or more identity with the amino acid sequence of SEQ ID NO: 14,
(L-A3) an amino acid sequence in which one or several amino acids are deleted, substituted, inserted, and/or added in the amino acid sequence of SEQ ID NO: 14,

(E) an antibody against the chikungunya virus, including the following heavy chain variable region (3) and the light chain variable region (2),

(3) a heavy chain variable region including a polypeptide composed of the following amino acid sequence (H-E),

(H-E) the following amino acid sequence (H-E1), (H-E2), or (H-E3),
(H-E1) an amino acid sequence of SEQ ID NO: 28,
(H-E2) an amino acid sequence having 80% or more identity with the amino acid sequence of SEQ ID NO: 28,
(H-E3) an amino acid sequence in which one or several amino acids are deleted, substituted, inserted, and/or added in the amino acid sequence of SEQ ID NO: 28.

9. The immunochromatographic analysis device according to any one of the above 1 to 6, wherein

the labeling substance retaining part further contains the following antibody (D) or an antigen-binding fragment thereof, and

the detection part further contains the following antibody (E) or an antigen-binding fragment thereof,

(D) an antibody against the chikungunya virus, including the following heavy chain (1) and the following light chain (2),

(1) a heavy chain including a polypeptide composed of the following amino acid sequence (HD),

(HD) the following amino acid sequence (HD1), (HD2), or (HD3),
(HD1) an amino acid sequence of SEQ ID NO: 29,
(HD2) an amino acid sequence having 80% or more identity with the amino acid sequence of SEQ ID NO: 29,
(HD3) an amino acid sequence in which one or several amino acids are deleted, substituted, inserted, and/or added in the amino acid sequence of SEQ ID NO: 29,

(2) a light chain including a polypeptide composed of the following amino acid sequence (LA),

(LA) the following amino acid sequence (LA1), (LA2), or (LA3),
(LA1) an amino acid sequence of SEQ ID NO: 18,
(LA2) an amino acid sequence having 80% or more identity with the amino acid sequence of SEQ ID NO: 18,
(LA3) an amino acid sequence in which one or several amino acids are deleted, substituted, inserted, and/or added in the amino acid sequence of SEQ ID NO: 18,

(E) an antibody against the chikungunya virus, including the following heavy chain (3) and the light chain (2),

(3) a heavy chain including a polypeptide composed of the following amino acid sequence (HE),

(HE) the following amino acid sequence (HE1), (HE2), or (HE3),
(HE1) an amino acid sequence of SEQ ID NO: 30,
(HE2) an amino acid sequence having 80% or more identity with the amino acid sequence of SEQ ID NO: 30,
(HE3) an amino acid sequence in which one or several amino acids are deleted, substituted, inserted, and/or added in the amino acid sequence of SEQ ID NO: 30.

10. The immunochromatographic analysis device according to any one of the above 7 to 9, wherein the antibody (D) or an antigen-binding fragment thereof, and the antibody (E) or an antigen-binding fragment thereof specifically bind to chikungunya virus having at least any one genotype of ECSA genotype, Asian genotype, and WA genotype.

11. The immunochromatographic analysis device according to any one of the above 7 to 10, wherein a content ratio (mass) of the antibody (A) or an antigen-binding fragment thereof to the antibody (D) or an antigen-binding fragment thereof in the labeling substance retaining part is 1:2 to 2:1.

12. The immunochromatographic analysis device according to any one of the above 1 to 11, wherein a sample to be loaded in the sample loading part is any one of the whole blood, serum, plasma, semen, and spinal fluid of a chikungunya virus infected individual.

13. An immunochromatographic analysis kit, including the immunochromatographic analysis device according to any one of the above 1 to 12, and a specimen diluent for diluting and developing a specimen.

14. An immunochromatographic analysis method for detecting chikungunya virus contained in a specimen using the immunochromatographic analysis kit according to the above 13, including the following steps (1) to (4):

(1) a step of loading a specimen-containing liquid obtained by diluting a specimen with a specimen diluent in the sample loading part;

(2) a step of allowing the antibody (A) or an antigen-binding fragment thereof held by the labeling substance retaining part to recognize the chikungunya virus;

(3) a step of developing the specimen and the antibody (A) or an antigen-binding fragment thereof in the chromatographic medium part as a mobile phase; and

(4) a step of detecting the chikungunya virus in the developed mobile phase with the antibody (B) or an antigen-binding fragment thereof and the antibody (C) or an antigen-binding fragment thereof contained in the detection part.

### Advantageous Effects of Invention

By using the immunochromatographic analysis device according to the present invention, even various chikungunya viruses with different genotypes, and chikungunya virus in which a part of the structural protein is mutated can be detected promptly and with high sensitivity. In addition, cross-reactivity with another virus (for example, Sindbis virus) other than the chikungunya virus is low, and the chikungunya virus can be specifically detected.

### BRIEF DESCRIPTION OF DRAWINGS

FIG. 1 is a cross-sectional view for illustrating a structure of an immunochromatographic analysis device of an embodiment of the present invention.

FIG. 2 is a graph showing the results of measuring chikungunya viruses (ECSA genotype and Asian genotype) using immunochromatographic analysis devices of Examples of the present invention and Comparative Example.

FIG. 3 is a graph showing the results of measuring chikungunya virus (WA genotype) using immunochromatographic analysis devices of Examples of the present invention and Comparative Example.

FIG. 4 is a graph showing the results of measuring wild type (WT) and mutant type (MT) chikungunya viruses (ECSA genotype) using immunochromatographic analysis devices of Examples of the present invention and Comparative Example.

FIG. 5 is a graph showing the results of measuring wild type (WT) and mutant type (MT) chikungunya viruses (Asian genotype) using immunochromatographic analysis devices of Examples of the present invention and Comparative Example.

### DESCRIPTION OF EMBODIMENTS

The immunochromatographic analysis device according to the present invention is characterized in that the labeling substance retaining part contains the antibody (A) or an antigen-binding fragment thereof, and the detection part contains the antibody (B) or an antigen-binding fragment thereof, and the antibody (C) or an antigen-binding fragment thereof as described above.

The immunochromatographic analysis device according to the present invention is configured such that by incorporating the antibodies or antigen-binding fragments thereof in the specific combination described above in the labeling substance retaining part and the detection part, even various chikungunya viruses with different genotypes, or chikungunya virus in which a part of the structural protein is mutated can be detected promptly and with high sensitivity, and further, cross-reactivity with another virus (for example, Sindbis virus) other than the chikungunya virus is low, and the chikungunya virus can be specifically detected.

Hereinafter, modes for carrying out the present invention will be described.

(Specimen)

The origin of a specimen (hereinafter sometimes also referred to as a specimen sample or simply a sample) that can be applied to the immunochromatographic analysis device according to the present invention is not particularly limited. For example, a human, a non-human animal other than a human, and the like are exemplified. Examples of the non-human animal include mammals such as a mouse, a rat, a dog, a monkey, a rabbit, sheep, and a horse, and arthropods such as Aedes aegypti and Aedes albopictus as a vector animal as described above.

The type of the specimen is not particularly limited, and for example, a biological sample such as a body fluid, urine, body fluid-derived cells, an organ, a tissue, or cells separated from a living body is exemplified. Examples of the body fluid include blood, a body cavity fluid such as synovial fluid, lymph fluid, tissue fluid, and the like, and specific examples include whole blood, serum, plasma, semen, spinal fluid, and the like. The biological sample is preferably whole blood, serum, plasma, semen, spinal fluid, and more preferably serum or plasma.

As the sample, for example, a collected sample may be used as it is in the present invention, or may be used after performing another treatment such as dilution with a liquid or the like. The liquid is not particularly limited, and for example, water, physiological saline, a buffer solution, a culture medium, and the like are exemplified. Further, the sample may be subjected to, for example, an acid treatment in advance. By doing this, for example, when chikungunya virus in the sample and an antibody or the like form an antigen-antibody complex, the antibody or the like and the chikungunya virus can be dissociated, so that the chikungunya virus can be detected with higher sensitivity. The acid used in the acid treatment is not particularly limited, and examples thereof include hydrochloric acid and the like.

(Antibodies or Antigen-Binding Fragments Thereof)

In the immunochromatographic analysis device according to the present invention, the labeling substance retaining part contains the antibody (A) or an antigen-binding fragment thereof, and the detection part contains the antibody (B) or an antigen-binding fragment thereof, and the antibody (C) or an antigen-binding fragment thereof as described above.

The antibody or the like or an antigen-binding fragment thereof (hereinafter also referred to as antibody or the like) specifically binds to chikungunya virus having at least any one genotype of ESCA genotype CHIKV, WA genotype CHIKV, and Asian genotype CHIKV. The ESCA genotype CHIKV, the WA genotype CHIKV, and the Asian genotype CHIKV can be classified with reference to, for example, the below-mentioned Reference Literature 1.

More specifically, the antibody or the like binds to, for example, an envelope glycoprotein (hereinafter also referred to as “Env protein”) of ESCA genotype CHIKV, an Env protein of WA genotype CHIKV, and an Env protein of Asian genotype CHIKV. The Env protein means, for example, a CHIKV envelope protein. The Env protein is constituted by, for example, 6K-E1 protein, E2 protein, and E3 protein, and therefore is also referred to as E3-E2-6K-E1 protein. For the amino acid sequence of the CHIKV Env protein, for example, information registered in an existing database can be referred to.

In a specific example, as the ESCA genotype CHIKV-derived Env protein, for example, the following amino acid sequence (SEQ ID NO: 31) from position 268 to position 1247 in the amino acid sequence registered under NCBI accession number BAP74220.1 is exemplified. As the WA genotype CHIKV-derived Env protein, for example, the following amino acid sequence (SEQ ID NO: 32) from position 268 to position 1247 in the amino acid sequence registered under NCBI accession number AAU43881.1 is exemplified. As the Asian genotype CHIKV-derived Env protein, for example, the following amino acid sequence (SEQ ID NO: 33) from position 268 to position 1247 in the amino acid sequence registered under NCBI accession number ADG95938.1 is exemplified.

Reference Literature 1: Aekkachai Tuekprakhon et. al., “Variation at position 350 in the Chikungunya virus 6K-E1 protein determines the sensitivity of detection in a rapid E1-antigen test”, Scientific Report, vol. 8, Article Number: 1094, 2018

ESCA genotype CHIKV-derived Env protein
(SEQ ID NO: 31)
MCLLANTTFPCSQPPCTPCCYEKEPEETLRMLEDNVMRPGYYQLLQASLT
CSPHRQRRSTKDNFNVYKATRPYLAHCPDCGEGHSCHSPVALERIRNEAT
DGTLKIQVSLQIGIKTDDSHDWTKLRYMDNHMPADAERAGLFVRTSAPCT
ITGTMGHFILARCPGKETLTVGFTDSRKISHSCTHPFHHDPPVIGREKFH
SRPQHGKELPCSTYVQSTAATTEEIEVHMPPDTPDRTLMSQQSGNVKITV
NGQTVRYKCNCGGSNEGLTTTDKVINNCKVDQCHAAVTNHKKWQYNSPLV
PRNAELGDRQKIHIPFPLANVTCRVPKARNPTVTYGKNQVIMLLYPDHPT
LLSYRNMGEEPNYQEEWVMHKKEVVLTVPTEGLEVTWGNNEPYKYWPQLS
TNGTAHGHPHEIILYYYELYPTMTVVVVSVATFILLSMVGMAAGMCMCAR
RRCITPYELTPGATVPFLLSLICCIRTAKAATYQEAAIYLWNEQQPLFWL
QALIPLAALIVLCNCLRLLPCCCKTLAFLAVMSVGAHTVSAYEHVTVIPN
TVGVPYKTLVNRPGYSPMVLEMELLSVTLEPTLSLDYITCEYKTVIPSPY
VKCCGTAECKDKNLPDYSCKVFTGVYPFMWGGAYCFCDAENTQLSEAHVE
KSESCKTEFASAYRAHTASASAKLRVLYQGNNITVYAYANGDHAVTVKDA
KFIVGPMSSAWTPFDNKIVVYKGDVYNMDYPPFGAGRPGQFGDIQSRTPE
SKDVYANTQLVLQRPAVGTVHVPYSQAPSGFKYWLKERGASLQHTAPFGC
QIATNPVRAVNCAVGNMPISIDIPEAAFTRVVDAPSLTDMSCEVPACTHS
SDFGGVAIIKYAASKKGKCAVHSMTNAVTIREAEIEVEGNSQLQISFSTA
LASAEFRVQVCSTQVHCAAECHPPKDHIVNYPASHTTLGVQDISATAMSW
VQKITGGVGLVVAVAALILIVVLCVSFSRH
WA genotype CHIKV-derived Env protein
(SEQ ID NO: 32)
LCLLANTTFPCSQPPCTPCCYEKEPESTLRMLEDNVMRPGYYQLLKASLT
CSPHRQRRSTKDNFNVYKATRPYLAHCPDCGEGHSCHSPIALERIRNEAT
DGTLKIQVSLQIGIKTDDSHDWTKLRYMDSHTPADAERAGLLVRTSAPCT
ITGTMGFFILARCPKGETLTVGFTDSRKISHTCTHPFHHEPPVIGRERGH
SRPQHGKELPCSTYVQSTAATAEEIEVHMPPDTPDRTLMTQQSGNVKITV
NGQTVRYKCNCGGSNEGLTTTDKVINNCKIDQCHAAVTNHKNWQYNSPLV
PRNAELGDRKGKIHIPFPLANVTCRVPKARNPTVTYGKNQVTMLLYPDHP
TLLSRYNMGQEPNYHEEWVTHKKEVTLTVPTEGLEVTWGNNEPYKYWPQM
STNGTAHGHPHEIILYYYELYPTMTVVIVSVASFVLLSMVGTAVGMCVCA
RRRCITPYELTPGATVPFLLSLLCCVRTTKAATYYEAAAYLWNEQQPLFW
LQALIPLAALIVLCNCLKLLPCCCKTLAFLAVMSIGAHTVSAYEHVTVIP
NTVGVPYKTLVNRPGYSPMVLEMELQSVTLEPTLSLDYITCEYKTVIPSP
YVKCCGTAECKDKSLPDYSCKVFTGVYPFMWGGAYCFCDAENTQLSEAHV
EKSESCKTEFASAYRAHTASASAKLRVLYQGNNITVAAYANGDHAVTVKD
AKFVVGPMSSAWTPFDNKIVVYKGDVYNMDYPPFGAGRPGQFGDIQSRTP
ESKDVYANTQLVLQRPAAGTVHVPYSQAPSGFKYWLKERGASLQHTAPFG
CIQATNPVRAVNCAVGNIPISIDIPDAAFTRVVDAPSVTDMSCEVPACTH
SSDFGGVAIIKYTASKKGKCAVHSMTNAVTIREADVEVEGNSQLQISFST
ALASAEFRVQVCSTQVHCAAACHPPKDHIVNYPASHTTLGVQDISTTAMS
WVQKITGGVGLIVAVAALILIVVLCVSFSRH
Asian genotype CHIKV-derived Env protein
(SEQ ID NO: 33)
MCLLANTTFPCSQPPCTPCCYEKEPEKTLRMLEDNVMSPGYYQLLQASLT
CSPRRQRRSIKDNFNVYKATRPYLAHCPDCGEGHSCHSPVALERIRNEAT
DGTLKIQVSLQIGIKTDDSHDWTKLRYMDNHMPADAERAGLFVRTSAPCT
ITGTMGHFILARCPKGETLTVGFTDGRKISHSCTHPFHHDPPVIGREKFH
SRPQHGRELPCSTYAQSTAATAEEIEVHMPPDTPDRTLMSQQSGNVKITV
NSQTVRYKCNCGDSNEGLTTTDKVINNCKVDQCHAAVTNHKKWQYNSPLV
PRNAELGDRKGKVHIPFPLANVTCRVPKARNPTVTYGKNQVIMLLYPDHP
TLLSYRNMGEEPNYQEEWVTHKKEIRLTVPTEGLEVTWGNNEPYKYWPQL
STNGTAHGHPHEIILYYYELYPTMTVVVVSVASFVLLSMVGVAVGMCMCA
RRRCITPYELTPGATVPFLLSLICCIRTAKAATYQEAAVYLWNEQQPLFW
LQALIPLAALIVLCNCLRLLPCCCKTLTFLAVLSVGAHTVSAYEHVTVIP
NTVGVPYKTLVNRPGYSPMVLEMELLSVTLEPTLSLDYITCEYKTVIPSP
YVKCCGTAECKDKSLPDYSCKVFTGVYPFMWGGAYCFCDTENTQLSEAHV
EKSESCKTEFASAYRAHTASASAKLRVLYQGNNVTVSAYANGDHAVTVKD
AKFIVGPMSSAWTPFDNKIVVYKGDVYNMDYPPFGAGRPGQFGDIQSRTP
ESEDVYANTQLVLQRPSAGTVHYPYSQAPSGFKYWLKERGASLQHTAPFG
CQIATNPVRAMNCAVGNMPISIDIPDAAFTRVVDAPSLTDMSCEVSACTH
SSDFGGVAIIKYAASKKGKCAVHSMTNAVTIREAEIEVEGNSQLQISFST
ALASAEFRVQVCSTQVHCAAECHPPKDHIVNYPASHTTLGVQDISATAMS
WVQKITGGVGLVVAVAALILIVVLCVSFSRH

The antibody or the like not only includes an antibody that binds to CHIKV, more specifically, a protein composed of the full-length amino acid sequence of the Env protein, but also includes the meaning of, for example, an antibody that binds to a peptide fragment of the Env protein. Further, the Env protein includes, for example, a mutant Env protein (E350D) in which an amino acid (in SEQ ID NO: 31, glutamic acid (E) indicated by an underline) corresponding to an amino acid at position 826 in the amino acid sequence of SEQ ID NO: 31 (at position 284 of the E1 protein and at position 350 of the 6K-E1 protein) is substituted with aspartic acid (D). In addition, the Env protein includes, for example, a mutant Env protein (D350E) in which an amino acid (in SEQ ID NO: 32 or 33, aspartic acid (D) indicated by an underline) corresponding to an amino acid at position 826 in the amino acid sequence of SEQ ID NO: 32 or 33 (at position 284 of the E1 protein and at position 350 of the 6K-E1 protein) is substituted with glutamic acid (E). Hereinafter, unless otherwise specified, the Env protein not only includes, for example, the Env protein, the mutant Env protein (E350D), or the mutant Env protein (D350E), each of which is composed of the full-length amino acid sequence, but also includes the meaning of a peptide fragment thereof.

The antibody or the like may be, for example, a so-called “antibody” whose molecular structure is an immunoglobulin, or may be an antigen-binding fragment thereof. The antibody or the like may have the heavy chain variable region and the light chain variable region described above. In the case of an antibody, for example, its immunoglobulin class and isotype are not particularly limited. Examples of the immunoglobulin class include IgG, IgM, IgA, IgD, IgE, and the like. Examples of the IgG include IgG1, IgG2, IgG3, IgG4, and the like.

The antibody may be, for example, a monoclonal antibody, a polyclonal antibody, a recombinant antibody, a human (for example, fully human) antibody, a humanized antibody, a chimeric antibody, a multispecific antibody, or the like.

The “antigen-binding fragment” in the present invention is a portion of the antibody, for example, a partial fragment, and also means a fragment that recognizes (binds to) the chikungunya virus. Examples of the antigen-binding fragment include a Fab, a Fab′, a (Fab′)₂, a variable region fragment (Fv), a disulfide bond Fv, a single chain Fv (scFv), a bispecific antibody and polymers thereof, and the like.

The antibody or the like may not only have the heavy chain variable region and the light chain variable region described above, but also have, for example, a constant region, and the constant region is, for example, a human constant region or a mouse constant region. In the case of an antibody (immunoglobulin), the heavy chain constant region includes, for example, regions called CH1, CH2, and CH3, and the light chain constant region includes, for example, a region called CL. When the antibody or the like in the present invention has the constant region, for example, the heavy chain variable region binds to at least one of CH1, CH2, and CH3, and the light chain variable region binds to the CL, and the heavy chain variable region directly binds to, for example, CH1.

In general, each of a heavy chain and a light chain of an antibody molecule has three complementarity determining regions (CDRs). The CDR is also referred to as a hypervariable domain. The CDR is a region having a particularly high primary structure variability even in the variable region of the heavy chain and the light chain, and is usually separated at three locations on the primary structure. In the present invention, the three CDRs in the heavy chain are represented by heavy chain CDR1 (CDRH1), heavy chain CDR2 (CDRH2), and heavy chain CDR3 (CDRH3) from the amino-terminal side in the amino acid sequence of the heavy chain. The three CDRs in the light chain are represented by light chain CDR1 (CDRL1), light chain CDR2 (CDRL2), and light chain CDR3 (CDRL3) from the amino-terminal side in the amino acid sequence of the light chain. These regions are close to each other on the steric structure and determine the specificity for the antigen to which the antibody binds.

Hereinafter, the antibody (A) will be described.

In an embodiment of the present invention, the antibody (A) is an antibody against chikungunya virus, including the following heavy chain variable region (1) and the following light chain variable region (2).

(1) a heavy chain variable region including a heavy chain complementarity determining region (CDRH) 1, CDRH2, and CDRH3,

CDRH1 being a polypeptide containing the following amino acid sequence (H1-A),

CDRH2 being a polypeptide containing the following amino acid sequence (H2-A), and

CDRH3 being a polypeptide containing the following amino acid sequence (H3-A)

(H1-A) the following amino acid sequence (H1-A1), (H1-A2), or (H-A3)
(H1-A1) an amino acid sequence of SEQ ID NO: 1
(H1-A2) an amino acid sequence having 80% or more identity with the amino acid sequence of SEQ ID NO: 1
(H1-A3) an amino acid sequence in which one or several amino acids are deleted, substituted, inserted, and/or added in the amino acid sequence of SEQ ID NO: 1

(H2-A) the following amino acid sequence (H2-A1), (H2-A2), or (H2-A3)
(H2-A1) an amino acid sequence of SEQ ID NO: 2
(H2-A2) an amino acid sequence having 80% or more identity with the amino acid sequence of SEQ ID NO: 2
(H2-A3) an amino acid sequence in which one or several amino acids are deleted, substituted, inserted, and/or added in the amino acid sequence of SEQ ID NO: 2

(H3-A) the following amino acid sequence (H3-A1), (H3-A2), or (H3-A3)
(H3-A1) an amino acid sequence of SEQ ID NO: 3
(H3-A2) an amino acid sequence having 80% or more identity with the amino acid sequence of SEQ ID NO: 3
(H3-A3) an amino acid sequence in which one or several amino acids are deleted, substituted, inserted, and/or added in the amino acid sequence of SEQ ID NO: 3

(2) a light chain variable region including a light chain complementarity determining region (CDRL) 1, CDRL2, and CDRL3,

CDRL1 being a polypeptide containing the following amino acid sequence (L1-A),

CDRL2 being a polypeptide containing the following amino acid sequence (L2-A), and

CDRL3 being a polypeptide containing the following amino acid sequence (L3-A)

(L1-A) the following amino acid sequence (L1-A1), (L1-A2), or (L1-A3)
(L1-A1) an amino acid sequence of SEQ ID NO: 4
(L1-A2) an amino acid sequence having 80% or more identity with the amino acid sequence of SEQ ID NO: 4
(L1-A3) an amino acid sequence in which one or several amino acids are deleted, substituted, inserted, and/or added in the amino acid sequence of SEQ ID NO: 4

(L2-A) the following amino acid sequence (L2-A1), (L2-A2), or (L2-A3)
(L2-A1) an amino acid sequence of SEQ ID NO: 5
(L2-A2) an amino acid sequence having 80% or more identity with the amino acid sequence of SEQ ID NO: 5
(L2-A3) an amino acid sequence in which one or several amino acids are deleted, substituted, inserted, and/or added in the amino acid sequence of SEQ ID NO: 5

(L3-A) the following amino acid sequence (L3-A1), (L3-A2), or (L3-A3)
(L3-A1) an amino acid sequence of SEQ ID NO: 6
(L3-A2) an amino acid sequence having 80% or more identity with the amino acid sequence of SEQ ID NO: 6
(L3-A3) an amino acid sequence in which one or several amino acids are deleted, substituted, inserted, and/or added in the amino acid sequence of SEQ ID NO: 6

In the respective CDRs, the “identity” is, for example, the degree of identity when sequences to be compared are appropriately subjected to alignment, and means the appearance ratio (%) of the exact matching of amino acids between the sequences. The “identity” in each case is, for example, 80% or more, 85% or more, 90% or more, 95% or more, 96% or more, 97% or more, 98% or more, or 99% or more. The identity can be calculated with default parameters using analysis software such as BLAST or FASTA (hereinafter the same shall apply).

In the respective CDRs, “one or several” related to substitution or the like in each case is, for example, 1 to 5, 1 to 4, 1 to 3, 1 or 2, or 1.

The amino acid substitution may be, for example, a conservative substitution (hereinafter the same shall apply). The conservative substitution means substitution of one or several amino acids with other amino acids and/or amino acid derivatives so as not to substantially alter the function of the protein. It is preferred that the “amino acid to be substituted for another” and the “amino acid to be substituted with another” have, for example, similar properties and/or functions. Specifically, for example, it is preferred that chemical properties such as hydrophobicity and hydrophilicity index (hydropathy), polarity, and charge, or physical properties such as secondary structure are similar. Amino acids or amino acid derivatives having similar properties and/or functions are, for example, known in the technical field. As specific examples, nonpolar amino acids (hydrophobic amino acids) include alanine, valine, isoleucine, leucine, proline, tryptophan, phenylalanine, methionine, and the like, polar amino acids (neutral amino acids) include glycine, serine, threonine, tyrosine, glutamine, asparagine, cysteine, and the like, positively charged amino acids (basic amino acids) include arginine, histidine, lysine, and the like, and negatively charged amino acids (acidic amino acids) include aspartic acid, glutamic acid, and the like.

In an embodiment of the present invention, the antibody (A) is an antibody against chikungunya virus, including the following heavy chain variable region (1) and the following light chain variable region (2).

(1) a heavy chain variable region including a polypeptide composed of the following amino acid sequence (H-A)

(H-A) the following amino acid sequence (H-A1), (H-A2), or (H-A3)
(H-A1) an amino acid sequence of SEQ ID NO: 13
(H-A2) an amino acid sequence having 80% or more identity with the amino acid sequence of SEQ ID NO: 13
(H-A3) an amino acid sequence in which one or several amino acids are deleted, substituted, inserted, and/or added in the amino acid sequence of SEQ ID NO: 13

(2) a light chain variable region including a polypeptide composed of the following amino acid sequence (L-A)

(L-A) the following amino acid sequence (L-A1), (L-A2), or (L-A3)
(L-A1) an amino acid sequence of SEQ ID NO: 14
(L-A2) an amino acid sequence having 80% or more identity with the amino acid sequence of SEQ ID NO: 14
(L-A3) an amino acid sequence in which one or several amino acids are deleted, substituted, inserted, and/or added in the amino acid sequence of SEQ ID NO: 14

In the polypeptide of the heavy chain variable region and the polypeptide of the light chain variable region, the “identity” in each case is, for example, 80% or more, 85% or more, 90% or more, 95% or more, 96% or more, 97% or more, 98% or more, or 99% or more.

In the polypeptide of the heavy chain variable region and the polypeptide of the light chain variable region, “one or several” related to substitution or the like in each case is, for example, 1 to 20, 1 to 15, 1 to 10, 1 to 9, 1 to 8, 1 to 7, 1 to 6, 1 to 5, 1 to 4, 1 to 3, 1 or 2, or 1.

In an embodiment of the present invention, the antibody (A) is an antibody against chikungunya virus, including the following heavy chain (1) and the following light chain (2).

(1) a heavy chain including a polypeptide composed of the following amino acid sequence (HA)

(HA) the following amino acid sequence (HA1), (HA2), or (HA3)
(HA1) an amino acid sequence of SEQ ID NO: 17
(HA2) an amino acid sequence having 80% or more identity with the amino acid sequence of SEQ ID NO: 17
(HA3) an amino acid sequence in which one or several amino acids are deleted, substituted, inserted, and/or added in the amino acid sequence of SEQ ID NO: 17

(2) a light chain including a polypeptide composed of the following amino acid sequence (LA)

(LA) the following amino acid sequence (LA1), (LA2), or (LA3)
(LA1) an amino acid sequence of SEQ ID NO: 18
(LA2) an amino acid sequence having 80% or more identity with the amino acid sequence of SEQ ID NO: 18
(LA3) an amino acid sequence in which one or several amino acids are deleted, substituted, inserted, and/or added in the amino acid sequence of SEQ ID NO: 18

In the polypeptide of the heavy chain and the polypeptide of the light chain, the “identity” in each case is, for example, 80% or more, 85% or more, 90% or more, 95% or more, 96% or more, 97% or more, 98% or more, or 99% or more.

In the polypeptide of the heavy chain and the polypeptide of the light chain, “one or several” related to substitution or the like in each case is, for example, 1 to 80, 1 to 60, 1 to 40, and 1 to 30, 1 to 20, 1 to 15, 1 to 10, 1 to 9, 1 to 8, 1 to 7, 1 to 6, 1 to 5, 1 to 4, 1 to 3, 1 or 2, or 1.

Next, the antibody (B) will be described.

In an embodiment of the present invention, the antibody (B) is an antibody against chikungunya virus, including the following heavy chain variable region (3) and the above light chain variable region (2) having the respective CDRs.

(3) a heavy chain variable region including a heavy chain complementarity determining region (CDRH) 1, CDRH2, and CDRH3,

CDRH1 being a polypeptide containing the following amino acid sequence (H1-B),

CDRH2 being a polypeptide containing the following amino acid sequence (H2-B), and

CDRH3 being a polypeptide containing the following amino acid sequence (H3-B)

(H1-B) the following amino acid sequence (H1-B1), (H1-B2), or (H1-B3)
(H1-B1) an amino acid sequence of SEQ ID NO: 7
(H1-B2) an amino acid sequence having 80% or more identity with the amino acid sequence of SEQ ID NO: 7
(H1-B3) an amino acid sequence in which one or several amino acids are deleted, substituted, inserted, and/or added in the amino acid sequence of SEQ ID NO: 7

(H2-B) the following amino acid sequence (H2-B1), (H2-B2), or (H2-B3)
(H2-B1) an amino acid sequence of SEQ ID NO: 8
(H2-B2) an amino acid sequence having 80% or more identity with the amino acid sequence of SEQ ID NO: 8
(H2-B3) an amino acid sequence in which one or several amino acids are deleted, substituted, inserted, and/or added in the amino acid sequence of SEQ ID NO: 8

(H3-B) the following amino acid sequence (H3-B1), (H3-B2), or (H3-B3)
(H3-B1) an amino acid sequence of SEQ ID NO: 9
(H3-B2) an amino acid sequence having 80% or more identity with the amino acid sequence of SEQ ID NO: 9
   (H3-B3) an amino acid sequence in which one or several amino acids are deleted, substituted, inserted, and/or added in the amino acid sequence of SEQ ID NO: 9

In the respective CDRs, the “identity” in each case is, for example, 80% or more, 85% or more, 90% or more, 95% or more, 96% or more, 97% or more, 98% or more, or 99% or more.

In the respective CDRs, “one or several” related to substitution or the like in each case is, for example, 1 to 5, 1 to 4, 1 to 3, 1 or 2, or 1.

In an embodiment of the present invention, the antibody (B) is an antibody against chikungunya virus, including the following heavy chain variable region (3) and the above light chain variable region (2).

(3) a heavy chain variable region including a polypeptide composed of the following amino acid sequence (H-B)

(H-B) the following amino acid sequence (H-B1), (H-B2), or (H-B3)
(H-B1) an amino acid sequence of SEQ ID NO: 15
(H-B2) an amino acid sequence having 80% or more identity with the amino acid sequence of SEQ ID NO: 15
(H-B3) an amino acid sequence in which one or several amino acids are deleted, substituted, inserted, and/or added in the amino acid sequence of SEQ ID NO: 15

In the polypeptide of the heavy chain variable region and the polypeptide of the light chain variable region, the “identity” in each case is, for example, 80% or more, 85% or more, 90% or more, 95% or more, 96% or more, 97% or more, 98% or more, or 99% or more.

In the polypeptide of the heavy chain variable region and the polypeptide of the light chain variable region, “one or several” related to substitution or the like in each case is, for example, 1 to 20, 1 to 15, 1 to 10, 1 to 9, 1 to 8, 1 to 7, 1 to 6, 1 to 5, 1 to 4, 1 to 3, 1 or 2, or 1.

In an embodiment of the present invention, the antibody (B) is an antibody against chikungunya virus, including the following heavy chain (3) and the above light chain (2).

(3) a heavy chain including a polypeptide composed of the following amino acid sequence (HB)

(HB) the following amino acid sequence (HB1), (HB2), or (HB3)
(HB1) an amino acid sequence of SEQ ID NO: 19
(HB2) an amino acid sequence having 80% or more identity with the amino acid sequence of SEQ ID NO: 19
(HB3) an amino acid sequence in which one or several amino acids are deleted, substituted, inserted, and/or added in the amino acid sequence of SEQ ID NO: 19

In the polypeptide of the heavy chain and the polypeptide of the light chain, the “identity” in each case is, for example, 80% or more, 85% or more, 90% or more, 95% or more, 96% or more, 97% or more, 98% or more, or 99% or more.

In the polypeptide of the heavy chain and the polypeptide of the light chain, “one or several” related to substitution or the like in each case is, for example, 1 to 80, 1 to 60, 1 to 40, and 1 to 30, 1 to 20, 1 to 15, 1 to 10, 1 to 9, 1 to 8, 1 to 7, 1 to 6, 1 to 5, 1 to 4, 1 to 3, 1 or 2, or 1.

Next, the antibody (C) will be described.

In an embodiment of the present invention, the antibody (C) is an antibody against chikungunya virus, including the following heavy chain variable region (4) and the above light chain variable region (2) having the respective CDRs.

(4) a heavy chain variable region including CDRH1, CDRH2, and CDRH3,

CDRH1 being a polypeptide containing the following amino acid sequence (H1-C),

CDRH2 being a polypeptide containing the following amino acid sequence (H2-C), and

CDRH3 being a polypeptide containing the following amino acid sequence (H3-C)

(H1-C) the following amino acid sequence (H1-C1), (H1-C2), or (H1-C3)
(H1-C1) an amino acid sequence of SEQ ID NO: 10
(H1-C2) an amino acid sequence having 80% or more identity with the amino acid sequence of SEQ ID NO: 10
(H1-C3) an amino acid sequence in which one or several amino acids are deleted, substituted, inserted, and/or added in the amino acid sequence of SEQ ID NO: 10

(H2-C) the following amino acid sequence (H2-C1), (H2-C2), or (H2-C3)
(H2-C1) an amino acid sequence of SEQ ID NO: 11
(H2-C2) an amino acid sequence having 80% or more identity with the amino acid sequence of SEQ ID NO: 11
(H2-C3) an amino acid sequence in which one or several amino acids are deleted, substituted, inserted, and/or added in the amino acid sequence of SEQ ID NO: 11

(H3-C) the following amino acid sequence (H3-C1), (H3-C2), or (H3-C3)
(H3-C1) an amino acid sequence of SEQ ID NO: 12
(H3-C2) an amino acid sequence having 80% or more identity with the amino acid sequence of SEQ ID NO: 12
(H3-C3) an amino acid sequence in which one or several amino acids are deleted, substituted, inserted, and/or added in the amino acid sequence of SEQ ID NO: 12

In the respective CDRs, the “identity” in each case is, for example, 80% or more, 85% or more, 90% or more, 95% or more, 96% or more, 97% or more, 98% or more, or 99% or more.

In the respective CDRs, “one or several” related to substitution or the like in each case is, for example, 1 to 5, 1 to 4, 1 to 3, 1 or 2, or 1.

In an embodiment of the present invention, the antibody (C) is an antibody against chikungunya virus, including the following heavy chain variable region (4) and the above light chain variable region (2).

(4) a heavy chain variable region including a polypeptide composed of the following amino acid sequence (H-C)

(H-C) the following amino acid sequence (H-C1), (H-C2), or (H-C3)
(H-C1) an amino acid sequence of SEQ ID NO: 16
(H-C2) an amino acid sequence having 80% or more identity with the amino acid sequence of SEQ ID NO: 16
(H-C3) an amino acid sequence in which one or several amino acids are deleted, substituted, inserted, and/or added in the amino acid sequence of SEQ ID NO: 16

In the polypeptide of the heavy chain variable region and the polypeptide of the light chain variable region, the “identity” in each case is, for example, 80% or more, 85% or more, 90% or more, 95% or more, 96% or more, 97% or more, 98% or more, or 99% or more.

In the polypeptide of the heavy chain variable region and the polypeptide of the light chain variable region, “one or several” related to substitution or the like in each case is, for example, 1 to 20, 1 to 15, 1 to 10, 1 to 9, 1 to 8, 1 to 7, 1 to 6, 1 to 5, 1 to 4, 1 to 3, 1 or 2, or 1.

In an embodiment of the present invention, the antibody (C) is an antibody against chikungunya virus, including the following heavy chain (4) and the above light chain (2).

(4) a heavy chain including a polypeptide composed of the following amino acid sequence (HC)

(HC) the following amino acid sequence (HC1), (HC2), or (HC3)
(HC1) an amino acid sequence of SEQ ID NO: 20
(HC2) an amino acid sequence having 80% or more identity with the amino acid sequence of SEQ ID NO: 20
(HC3) an amino acid sequence in which one or several amino acids are deleted, substituted, inserted, and/or added in the amino acid sequence of SEQ ID NO: 20

In the polypeptide of the heavy chain and the polypeptide of the light chain, the “identity” in each case is, for example, 80% or more, 85% or more, 90% or more, 95% or more, 96% or more, 97% or more, 98% or more, or 99% or more.

In the polypeptide of the heavy chain and the polypeptide of the light chain, “one or several” related to substitution or the like in each case is, for example, 1 to 80, 1 to 60, 1 to 40, and 1 to 30, 1 to 20, 1 to 15, 1 to 10, 1 to 9, 1 to 8, 1 to 7, 1 to 6, 1 to 5, 1 to 4, 1 to 3, 1 or 2, or 1.

The antibody (A) or an antigen-binding fragment thereof, the antibody (B) or an antigen-binding fragment thereof, and the antibody (C) or an antigen-binding fragment thereof specifically bind to chikungunya virus having at least any one genotype of ECSA genotype, Asian genotype, and WA genotype.

Next, the antibody (D) will be described.

In an embodiment of the present invention, the antibody (D) is an antibody against chikungunya virus, including the following heavy chain variable region (1) and the following light chain variable region (2).

(1) a heavy chain variable region including a heavy chain complementarity determining region (CDRH) 1, CDRH2, and CDRH3,

CDRH1 being a polypeptide containing the following amino acid sequence (H1-D),

CDRH2 being a polypeptide containing the following amino acid sequence (H2-D), and

CDRH3 being a polypeptide containing the following amino acid sequence (H3-D)

(H1-D) the following amino acid sequence (H1-D1), (H1-D2), or (H1-D3)
(H1-D1) an amino acid sequence of SEQ ID NO: 21
(H1-D2) an amino acid sequence having 80% or more identity with the amino acid sequence of SEQ ID NO: 21
(H1-D3) an amino acid sequence in which one or several amino acids are deleted, substituted, inserted, and/or added in the amino acid sequence of SEQ ID NO: 21

(H2-D) the following amino acid sequence (H2-D1), (H2-D2), or (H2-D3)
(H2-D1) an amino acid sequence of SEQ ID NO: 22
(H2-D2) an amino acid sequence having 80% or more identity with the amino acid sequence of SEQ ID NO: 22
(H2-D3) an amino acid sequence in which one or several amino acids are deleted, substituted, inserted, and/or added in the amino acid sequence of SEQ ID NO: 22

(H3-D) the following amino acid sequence (H3-D1), (H3-D2), or (H3-D3)
(H3-D1) an amino acid sequence of SEQ ID NO: 23
(H3-D2) an amino acid sequence having 80% or more identity with the amino acid sequence of SEQ ID NO: 23
(H3-D3) an amino acid sequence in which one or several amino acids are deleted, substituted, inserted, and/or added in the amino acid sequence of SEQ ID NO: 23

(2) a light chain variable region including a light chain complementarity determining region (CDRL) 1, CDRL2, and CDRL3,

CDRL1 being a polypeptide containing the following amino acid sequence (L1-A),

CDRL2 being a polypeptide containing the following amino acid sequence (L2-A), and

CDRL3 being a polypeptide containing the following amino acid sequence (L3-A)

(L1-A) the following amino acid sequence (L1-A1), (L1-A2), or (L1-A3)
(L1-A1) an amino acid sequence of SEQ ID NO: 4
(L1-A2) an amino acid sequence having 80% or more identity with the amino acid sequence of SEQ ID NO: 4
(L1-A3) an amino acid sequence in which one or several amino acids are deleted, substituted, inserted, and/or added in the amino acid sequence of SEQ ID NO: 4

(L2-A) the following amino acid sequence (L2-A1), (L2-A2), or (L2-A3)
(L2-A1) an amino acid sequence of SEQ ID NO: 5
(L2-A2) an amino acid sequence having 80% or more identity with the amino acid sequence of SEQ ID NO: 5
(L2-A3) an amino acid sequence in which one or several amino acids are deleted, substituted, inserted, and/or added in the amino acid sequence of SEQ ID NO: 5

(L3-A) the following amino acid sequence (L3-A1), (L3-A2), or (L3-A3)
(L3-A1) an amino acid sequence of SEQ ID NO: 6
(L3-A2) an amino acid sequence having 80% or more identity with the amino acid sequence of SEQ ID NO: 6
(L3-A3) an amino acid sequence in which one or several amino acids are deleted, substituted, inserted, and/or added in the amino acid sequence of SEQ ID NO: 6

In the respective CDRs, the “identity” in each case is, for example, 80% or more, 85% or more, 90% or more, 95% or more, 96% or more, 97% or more, 98% or more, or 99% or more.

In the respective CDRs, “one or several” related to substitution or the like in each case is, for example, 1 to 5, 1 to 4, 1 to 3, 1 or 2, or 1.

In an embodiment of the present invention, the antibody (D) is an antibody against chikungunya virus, including the following heavy chain variable region (1) and the following light chain variable region (2).

(1) a heavy chain variable region including a polypeptide composed of the following amino acid sequence (H-D)

(H-D) the following amino acid sequence (H-D1), (H-D2), or (H-D3)
(H-D1) an amino acid sequence of SEQ ID NO: 27
(H-D2) an amino acid sequence having 80% or more identity with the amino acid sequence of SEQ ID NO: 27
(H-D3) an amino acid sequence in which one or several amino acids are deleted, substituted, inserted, and/or added in the amino acid sequence of SEQ ID NO: 27

(2) a light chain variable region including a polypeptide composed of the following amino acid sequence (L-D)

(L-D) the following amino acid sequence (L-D1), (L-D2), or (L-D3)
(L-D1) an amino acid sequence of SEQ ID NO: 14
(L-D2) an amino acid sequence having 80% or more identity with the amino acid sequence of SEQ ID NO: 14
(L-D3) an amino acid sequence in which one or several amino acids are deleted, substituted, inserted, and/or added in the amino acid sequence of SEQ ID NO: 14

In the polypeptide of the heavy chain variable region and the polypeptide of the light chain variable region, the “identity” in each case is, for example, 80% or more, 85% or more, 90% or more, 95% or more, 96% or more, 97% or more, 98% or more, or 99% or more.

In the polypeptide of the heavy chain variable region and the polypeptide of the light chain variable region, “one or several” related to substitution or the like in each case is, for example, 1 to 20, 1 to 15, 1 to 10, 1 to 9, 1 to 8, 1 to 7, 1 to 6, 1 to 5, 1 to 4, 1 to 3, 1 or 2, or 1.

In an embodiment of the present invention, the antibody (D) is an antibody against chikungunya virus, including the following heavy chain (1) and the following light chain (2).

(1) a heavy chain including a polypeptide composed of the following amino acid sequence (HD)

(HD) the following amino acid sequence (HD1), (HD2), or (HD3)
(HD1) an amino acid sequence of SEQ ID NO: 29
(HD2) an amino acid sequence having 80% or more identity with the amino acid sequence of SEQ ID NO: 29
(HD3) an amino acid sequence in which one or several amino acids are deleted, substituted, inserted, and/or added in the amino acid sequence of SEQ ID NO: 29

(2) a light chain including a polypeptide composed of the following amino acid sequence (LD)

(LD) the following amino acid sequence (LD1), (LD2), or (LD3)
(LD1) an amino acid sequence of SEQ ID NO: 18
(LD2) an amino acid sequence having 80% or more identity with the amino acid sequence of SEQ ID NO: 18
(LD3) an amino acid sequence in which one or several amino acids are deleted, substituted, inserted, and/or added in the amino acid sequence of SEQ ID NO: 18

In the polypeptide of the heavy chain and the polypeptide of the light chain, the “identity” in each case is, for example, 80% or more, 85% or more, 90% or more, 95% or more, 96% or more, 97% or more, 98% or more, or 99% or more.

In the polypeptide of the heavy chain and the polypeptide of the light chain, “one or several” related to substitution or the like in each case is, for example, 1 to 80, 1 to 60, 1 to 40, and 1 to 30, 1 to 20, 1 to 15, 1 to 10, 1 to 9, 1 to 8, 1 to 7, 1 to 6, 1 to 5, 1 to 4, 1 to 3, 1 or 2, or 1.

Next, the antibody (E) will be described.

In an embodiment of the present invention, the antibody (E) is an antibody against chikungunya virus, including the following heavy chain variable region (3) and the above light chain variable region (2) having the respective CDRs.

(3) a heavy chain variable region including a heavy chain complementarity determining region (CDRH) 1, CDRH2, and CDRH3,

CDRH1 being a polypeptide containing the following amino acid sequence (H1-E),

CDRH2 being a polypeptide containing the following amino acid sequence (H2-E), and

CDRH3 being a polypeptide containing the following amino acid sequence (H3-E)

(H1-E) the following amino acid sequence (H1-E1), (H1-E2), or (H1-E3)
(H1-E1) an amino acid sequence of SEQ ID NO: 24
(H1-E2) an amino acid sequence having 80% or more identity with the amino acid sequence of SEQ ID NO: 24
(H1-E3) an amino acid sequence in which one or several amino acids are deleted, substituted, inserted, and/or added in the amino acid sequence of SEQ ID NO: 24

(H2-E) the following amino acid sequence (H2-E1), (H2-E2), or (H2-E3)
(H2-E1) an amino acid sequence of SEQ ID NO: 25
(H2-E2) an amino acid sequence having 80% or more identity with the amino acid sequence of SEQ ID NO: 25
(H2-E3) an amino acid sequence in which one or several amino acids are deleted, substituted, inserted, and/or added in the amino acid sequence of SEQ ID NO: 25

(H3-E) the following amino acid sequence (H3-E1), (H3-E2), or (H3-E3)
(H3-E1) an amino acid sequence of SEQ ID NO: 26
(H3-E2) an amino acid sequence having 80% or more identity with the amino acid sequence of SEQ ID NO: 26
(H3-E3) an amino acid sequence in which one or several amino acids are deleted, substituted, inserted, and/or added in the amino acid sequence of SEQ ID NO: 26

In the respective CDRs, the “identity” in each case is, for example, 80% or more, 85% or more, 90% or more, 95% or more, 96% or more, 97% or more, 98% or more, or 99% or more.

In the respective CDRs, “one or several” related to substitution or the like in each case is, for example, 1 to 5, 1 to 4, 1 to 3, 1 or 2, or 1.

In an embodiment of the present invention, the antibody (E) is an antibody against chikungunya virus, including the following heavy chain variable region (3) and the above light chain variable region (2).

(3) a heavy chain variable region including a polypeptide composed of the following amino acid sequence (H-E)

(H-E) the following amino acid sequence (H-E1), (H-E2), or (H-E3)
(H-E1) an amino acid sequence of SEQ ID NO: 28
(H-E2) an amino acid sequence having 80% or more identity with the amino acid sequence of SEQ ID NO: 28
(H-E3) an amino acid sequence in which one or several amino acids are deleted, substituted, inserted, and/or added in the amino acid sequence of SEQ ID NO: 28

In the polypeptide of the heavy chain variable region and the polypeptide of the light chain variable region, the “identity” in each case is, for example, 80% or more, 85% or more, 90% or more, 95% or more, 96% or more, 97% or more, 98% or more, or 99% or more.

In the polypeptide of the heavy chain variable region and the polypeptide of the light chain variable region, “one or several” related to substitution or the like in each case is, for example, 1 to 20, 1 to 15, 1 to 10, 1 to 9, 1 to 8, 1 to 7, 1 to 6, 1 to 5, 1 to 4, 1 to 3, 1 or 2, or 1.

In an embodiment of the present invention, the antibody (E) is an antibody against chikungunya virus, including the following heavy chain (3) and the above light chain (2).

(3) a heavy chain including a polypeptide composed of the following amino acid sequence (HE)

(HE) the following amino acid sequence (HE1), (HE2), or (HE3)
(HE1) an amino acid sequence of SEQ ID NO: 30
(HE2) an amino acid sequence having 80% or more identity with the amino acid sequence of SEQ ID NO: 30
(HE3) an amino acid sequence in which one or several amino acids are deleted, substituted, inserted, and/or added in the amino acid sequence of SEQ ID NO: 30

In the polypeptide of the heavy chain and the polypeptide of the light chain, the “identity” in each case is, for example, 80% or more, 85% or more, 90% or more, 95% or more, 96% or more, 97% or more, 98% or more, or 99% or more.

In the polypeptide of the heavy chain and the polypeptide of the light chain, “one or several” related to substitution or the like in each case is, for example, 1 to 80, 1 to 60, 1 to 40, and 1 to 30, 1 to 20, 1 to 15, 1 to 10, 1 to 9, 1 to 8, 1 to 7, 1 to 6, 1 to 5, 1 to 4, 1 to 3, 1 or 2, or 1.

The antibody (D) or an antigen-binding fragment thereof, and the antibody (E) or an antigen-binding fragment thereof specifically bind to chikungunya virus having at least any one genotype of ECSA genotype, Asian genotype, and WA genotype.

In the present invention, the amino acid sequences of SEQ ID NOS: 1 to 30 are, for example, mouse-derived amino acid sequences.

A method for producing the antibody or the like is not particularly limited, and for example, the antibody or the like can be produced by genetic engineering based on the information of the amino acid sequence described above. Specifically, for example, the production can be carried out as follows. Note that the present invention is not limited to this example.

First, a vector containing a nucleic acid sequence encoding the amino acid sequence of each of the regions, the heavy chain, and/or the light chain of the antibody or the like is introduced into a host to obtain a transformant. Then, the transformant is cultured, and a fraction containing an antibody that binds to the chikungunya virus, specifically, the Env protein, is collected, and the antibody is isolated or purified from the obtained collected fraction.

Examples of the vector include a vector containing a nucleic acid sequence encoding the heavy chain variable region, a vector containing a nucleic acid sequence encoding the light chain variable region, a vector containing a nucleic acid sequence encoding the heavy chain, a vector containing a nucleic acid sequence encoding the light chain, and the like. The host is not particularly limited and may be any as long as it can introduce the vector and express the nucleic acid sequence in the vector. Examples of the host include mammalian cells such as HEK cells, CHO cells, COS cells, NSO cells, and SP2/0 cells. The method for introducing the vector into a host is not particularly limited, and a known method can be adopted.

A method for culturing the transformant is not particularly limited, and can be appropriately determined according to the type of the host. The fraction containing the antibody can be collected, for example, as a liquid fraction by homogenizing the cultured transformant. The isolation or purification of the antibody is not particularly limited, and a known method can be adopted.

In the present invention, the antibody is, for example, a monoclonal antibody. Examples of the monoclonal antibody include a monoclonal antibody obtained by immunization of an animal, a chimeric antibody, a humanized antibody, a human antibody (also referred to as a fully human antibody), and the like.

The chimeric antibody is an antibody in which a variable region of an antibody derived from an animal other than a human and a constant region of a human antibody are linked. The chimeric antibody can be produced, for example, as follows. First, with respect to a monoclonal antibody derived from an animal other than a human, a gene of a variable region (V region) that binds to chikungunya virus, specifically, the Env protein, is prepared, and the gene of the variable region and a gene of a constant region (C region) of a human antibody are linked, and the resultant is further linked to an expression vector. Then, cells transfected with the expression vector are cultured, and a target chimeric antibody secreted into the culture solution is collected. By doing this, the chimeric antibody can be prepared. The animal from which the gene of the variable region is derived is not particularly limited, and examples thereof include a rat, a mouse, and the like. The method for producing the chimeric antibody is not limited thereto, and the production can be carried out, for example, with reference to a known method such as a method described in JP-B-H3-73280.

The humanized antibody is an antibody in which only the CDR is derived from an animal other than a human and the other regions are derived from a human. The humanized antibody can be produced, for example, as follows. First, with respect to a monoclonal antibody derived from an animal other than a human, a gene of the CDR is prepared and grafted into a gene of a human antibody, for example, a constant region (CDR grafting), and further, the resultant is linked to an expression vector. Then, cells transfected with the expression vector are cultured, whereby the humanized antibody in which the target CDR is grafted is secreted in the culture solution. The humanized antibody can be prepared by collecting the secreted humanized antibody. The animal from which the CDR is derived is not particularly limited, and examples thereof include a rat, a mouse, and the like. The method for producing a humanized antibody is not limited thereto, and the production can be carried out, for example, with reference to a known method such as a method described in JP-T-H4-506458, JP-A-S62-296890, or the like.

The human antibody is an antibody in which all the regions are derived from a human. The human antibody can be prepared, for example, by introducing a human antibody gene into an animal other than a human. As the animal into which the human antibody gene is introduced, for example, a transgenic animal for producing a human antibody can be used. The type of the animal is not particularly limited, and examples thereof include a mouse and the like. As for the method for producing the human antibody, the production can be carried out with reference to a known method described in, for example, Nature Genetics, Vol. 7, pp. 13-21, 1994; Nature Genetics, Vol. 15, pp. 146-156, 1997; JP-T-H4-504365; JP-T-H7-509137; WO 1994/25585; Nature, Vol. 368, pp. 856-859, 1994; JP-T-H6-500233, or the like. Further, the human antibody can also be produced, for example, using a phage display method, and the production can be carried out with reference to a known method described in, for example, Marks, J. D. et al.: J. Mol. Biol., Vol. 222, pp. 581-597, 1991, or the like.

The antibody or the like can also be prepared, for example, by immunizing an animal with an antigen. Examples of the antigen include chikungunya virus, specifically, a protein composed of the full-length amino acid sequence of the Env protein or a peptide fragment thereof. It is preferred to carry out immunization with the antigen multiple times. In that case, the antigen to be used for the immunization each time is preferably, for example, chikungunya virus with a different genotype or the Env protein, or a peptide fragment thereof. The peptide fragment may be, for example, a peptide fragment composed only of an antigenic determinant (epitope) or a peptide fragment containing the antigenic determinant.

The monoclonal antibody obtained by immunization of the animal can be produced, for example, with reference to a known method such as a method described in Current Protocols in Molecular Biology (John Wiley & Sons (1987)), Antibodies: A Laboratory Manual, Ed. Harlow and David Lane, Cold Spring Harbor Laboratory (1988), or the like.

Specifically, for example, an animal is immunized with an antigen, and an antibody-producing cell collected from the immunized animal is fused with a myeloma cell lacking the ability to produce an autoantibody thereby producing a hybridoma. Subsequently, an antibody-producing cell is screened from the hybridoma, and a single hybridoma clone is prepared by cloning. Then, this hybridoma clone is administered to an animal, and the monoclonal antibody is purified from the obtained abdominal cavity. Alternatively, the hybridoma is cultured, and the monoclonal antibody is purified from the culture solution. In this manner, by producing the hybridoma clone, a monoclonal antibody with uniform specificity can be stably supplied.

The myeloma cell is preferably derived from, for example, a mouse, a rat, a human, or the like. The myeloma cell and the antibody-producing cell may be, for example, derived from the same species or different species, but are preferably derived from the same species.

(Immunochromatographic Analysis Device According to the Invention)

Next, an embodiment of an immunochromatographic analysis device according to the present invention will be described with reference to the drawings. In the present invention, “fix” means that the antibody or the like is placed on a support such as a membrane so that the antibody or the like does not migrate, and “hold” means that the antibody or the like is placed so that the antibody or the like can migrate in the support such as a membrane or on the surface thereof.

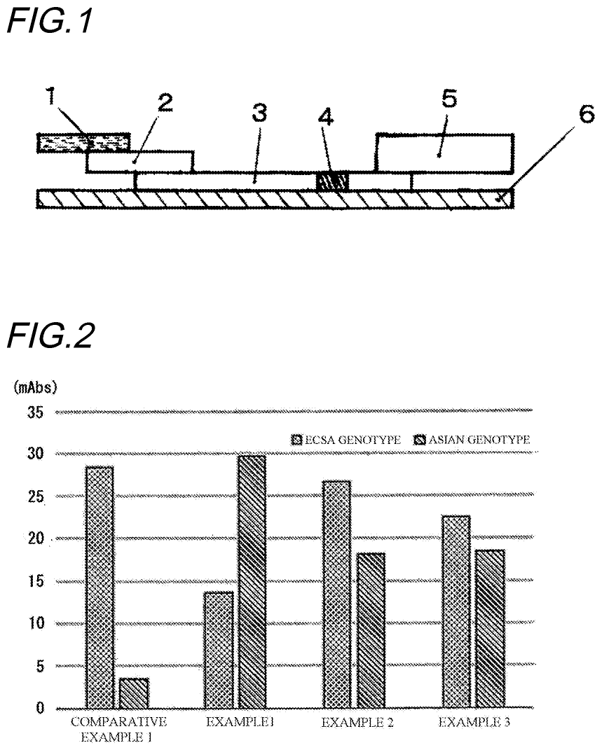

In an embodiment of the immunochromatographic analysis device according to the present invention, as shown in FIG. 1, the immunochromatographic analysis device is constituted by a sample loading part (1), a labeling substance retaining part (2), a chromatographic medium part (3), a detection part (4), an absorption part (5), and a backing sheet (6).

The sample loading part (1) is a part in which a specimen sample is loaded in the immunochromatographic analysis device. The sample loading part (1) can be constituted by a porous sheet having properties such that the specimen sample is rapidly absorbed but the holding power is low, and the specimen sample promptly migrates. Examples of the porous sheet include a cellulose filter paper, a glass fiber filter paper, polyurethane, polyacetate, cellulose acetate, nylon, a cotton cloth, and the like.

In the sample loading part (1), in order to promote an antigen-antibody reaction or suppress a non-specific reaction, a buffer solution, a salt such as casein sodium, a surfactant such as a nonionic surfactant, a cationic surfactant, or an anionic surfactant, a polymer compound such as polyvinylpyrrolidone (PVP), a polyanion, a nitrogen atom-containing vinyl-based water-soluble polymer, an antibacterial agent, a chelating agent, or the like can be incorporated. Among these, one type or two or more types may be incorporated.

As the surfactant, a surfactant having an HLB value of 13 to 17 can be preferably used. Examples thereof include Triton X-100 (trade name, polyethylene glycol mono-p-isooctylphenyl ether, HLB: 13.7), Tween 20 (trade name, polyoxyethylene sorbitan monolaurate, HLB: 16.7), Tween 80 (trade name, polyoxyethylene sorbitan monooleate, HLB: 15.0), Brij 35 (polyoxyethylene glycol dodecyl ether, HLB: 13.7), and the like, and one type or two or more types may be added. By incorporating a surfactant having an HLB value of 13 to 17 in the sample loading part, an antigen-antibody non-specific reaction in the labeling substance retaining part and the detection part can be suppressed, and the detection sensitivity to a detection target in the detection part can be increased.

The content of the surfactant per unit area of the sample loading part (1) is usually 0.25 μg/cm² to 8 μg/cm², preferably 0.5 μg/cm² to 5 μg/cm², and more preferably 1 μg/cm² to 4 μg/cm. When the content is within the above range, promotion of an antigen-antibody reaction or suppression of a non-specific reaction can be achieved.

Further, examples of the nitrogen atom-containing vinyl-based water-soluble polymer include polyvinylpyrrolidone (PVP), and the like. By incorporating a nitrogen atom-containing vinyl-based water-soluble polymer in the sample loading part, it is possible to enhance the signal by arbitrarily aggregating an insoluble carrier that has reacted with the antigen, and therefore, the detection sensitivity to a detection target in the detection part can be increased.

The content of the nitrogen atom-containing vinyl-based water-soluble polymer per unit area in the sample loading part (1) is usually 0.05 μg/cm² to 0.5 μg/cm², preferably 0.1 μg/cm² to 0.5 μg/cm², and more preferably 0.2 μg/cm² to 0.4 μg/cm. When the content is within the above range, promotion of an antigen-antibody reaction or suppression of a non-specific reaction can be achieved.

The labeling substance retaining part (2) contains the below-mentioned labeling substance, and the labeling substance is held in the labeling substance retaining part (2) as a labeled antibody or the like in which the labeling substance is bound to the antibody or the like. The type of the antibody or the like that binds to the labeling substance is not limited to one type, but includes at least the antibody (A) or an antigen-binding fragment thereof. When the specimen migrates in the labeling substance retaining part, the labeled antibody or the like held in the labeling substance retaining part and chikungunya virus in the specimen are bound to each other. In the labeling substance retaining part (2), a membrane of glass fiber or cellulose is usually used.

The content of the antibody (A) or an antigen-binding fragment thereof in the labeling substance retaining part is usually 0.01 μg to 1 μg, preferably 0.075 μg to 0.75 μg, and more preferably 0.1 μg to 0.5 μg. The content of the antibody (A) or an antigen-binding fragment thereof per unit area of the labeling substance retaining part is usually 0.01 μg/cm² to 1.8 μg/cm², preferably 0.13 μg/cm² to 1.4 μg/cm², and more preferably 0.15 μg/cm² to 1 μg/cm².

Further, the antibody that binds to the labeling substance preferably includes the antibody (D) or an antigen-binding fragment thereof. In that case, the content of the antibody (D) or an antigen-binding fragment thereof in the labeling substance retaining part is usually 0.05 μg to 1 μg, preferably 0.075 μg to 0.75 μg, and more preferably 0.1 μg to 0.5 μg. In addition, the content of the antibody (D) or an antigen-binding fragment thereof per unit area of the labeling substance retaining part is usually 0.01 μg/cm² to 1.8 μg/cm², preferably 0.13 μg/cm² to 1.4 μg/cm², and more preferably 0.15 μg/cm² to 1 μg/cm².

In addition, the mass ratio of the content of the antibody (A) or an antigen-binding fragment thereof to the content of the antibody (D) or an antigen-binding fragment thereof in the labeling substance retaining part is preferably 1:2 to 2:1, and more preferably 1:1.

As the labeling substance, an enzyme or the like is generally used, but it is preferred to use an insoluble carrier as the labeling substance because it is suitable for visually determining the presence of a detection target. As the insoluble carrier serving as the labeling substance, metal particles of gold, silver, platinum, or the like, metal oxide particles of iron oxide or the like, non-metallic particles of sulfur or the like, and latex particles composed of a synthetic polymer, or other insoluble carriers can be used. As described above, the insoluble carrier is a labeling substance suitable for visually determining the presence of a detection target, and is preferably colored so as to facilitate visual determination. The metal particles and the metal oxide particles are those exhibiting a specific natural color corresponding to the particle diameter by themselves, and the color can be used as a label.

As the insoluble carrier serving as the labeling substance, gold particles are particularly preferred because they are easy to detect and are difficult to aggregate, and non-specific color development is unlikely to occur. The average particle diameter of the gold particles is, for example, 10 nm to 250 nm, preferably 35 nm to 120 nm, and more preferably 40 nm to 80 nm. The average particle diameter can be determined, for example, by randomly measuring the projected area equivalent circle diameters of 100 particles using a projected photograph taken by a transmission electron microscope (TEM: manufactured by JEOL Ltd., JEM-2010) and performing calculation from the average thereof. The content of the gold particles in the labeling substance retaining part per unit area of the labeling substance retaining part is usually 0.006 μg/cm² to 0.42 μg/cm², preferably 0.01 μg/cm² to 0.3 μg/cm², and more preferably 0.01 μg/cm² to 0.2 μg/cm². When the content of the gold particles is within the above range, the labeled particles are developed while remaining in a dispersed state, and the sensitivity can be increased without inhibiting the recognition site of the antibody or the like.

The chromatographic medium part (3) is a developing part in a chromatograph. The chromatographic medium part (3) is an inert membrane composed of a microporous material exhibiting a capillary phenomenon. From the viewpoint of not having reactivity with a detection reagent, an immobilization reagent, a detection target, or the like, or from the viewpoint of improving the effect of the present invention, in the chromatographic medium part (3), for example, a membrane made of nitrocellulose or a membrane made of cellulose acetate can be preferably used. In particular, a membrane made of nitrocellulose is preferred. Note that a cellulose-based membrane, a nylon membrane, and a porous plastic cloth of polyethylene, polypropylene, or the like can also be used.

The membrane made of nitrocellulose only needs to contain nitrocellulose as a main component, and a membrane mainly composed of nitrocellulose such as a pure product or a mixed product of nitrocellulose can be used.

The membrane made of nitrocellulose can further contain a substance that promotes a capillary phenomenon. As the substance, a substance that lowers the surface tension of the membrane surface and brings about hydrophilicity is preferred. For example, a substance having an amphiphilic action, does not affect the migration of the detection target, and does not affect the development of the color of the labeling substance such as a saccharide, an amino acid derivative, a fatty acid ester, or any of a variety of synthetic surfactants or alcohols is preferred.

The membrane made of nitrocellulose is porous and exhibits a capillary phenomenon. The index of the capillary phenomenon can be confirmed by measuring a water absorption rate (water absorption time: capillary flow time). The water absorption rate affects detection sensitivity and an examination time.

The form and size of the chromatographic medium part (3) represented by a membrane made of nitrocellulose or a membrane made of cellulose acetate as described above are not particularly limited, and may be any form and size as long as they are appropriate in terms of the actual operation and the observation of the reaction result.

In order to further simplify the operation, it is preferred to provide a support made of a plastic or the like on the back face of the chromatographic medium part (3). The properties of the support are not particularly limited, but when observation of the measurement result is carried out by visual determination, the support preferably has a color that is not similar to the color brought about by the labeling substance, and is usually preferably colorless or white.

Further, on the chromatographic medium part (3), in order to prevent a decrease in the accuracy of analysis due to non-specific adsorption, the chromatographic medium part (3) can be subjected to a blocking treatment by a known method as needed. In general, proteins such as bovine serum albumin, skim milk, casein, and gelatin are preferably used for the blocking treatment. After such a blocking treatment, for example, washing may be performed using one surfactant or a combination of two or more surfactants such as Tween 20, Triton X-100, and SDS as needed.

The detection part (4) is formed at an arbitrary position on the chromatographic medium part (3), and is at least the antibody (B) or an antigen-binding fragment thereof and the antibody (C) or an antigen-binding fragment thereof are contained. Immobilization of the antibody to the detection part (4) can be carried out according to a conventional method.

In the detection part (4), chikungunya virus in the specimen that has passed on the chromatographic medium part as a mobile phase specifically reacts and binds so as to be interposed between the antibody fixed to the detection part (4) and the labeled antibody or the like in a sandwich-like form.

The content of the antibody (B) or an antigen-binding fragment thereof contained in the detection part (4) is usually 0.05 μg to 1 μg, preferably 0.2 μg to 0.7 μg, and more preferably 0.2 μg to 0.6 μg. Further, the content of the antibody (B) or an antigen-binding fragment thereof per unit area of the detection part (4) is usually 0.05 μg/cm² to 2 μg/cm², preferably 0.1 μg/cm² to 1 μg/cm², and more preferably 0.2 μg/cm² to 0.7 μg/cm².

The content of the antibody (C) or an antigen-binding fragment thereof contained in the detection part (4) is usually 0.1 μg to 1 μg, preferably 0.2 μg to 0.7 μg, and more preferably 0.3 μg to 0.6 μg. Further, the content of the antibody (C) or an antigen-binding fragment thereof per unit area of the detection part (4) is usually 0.15 μg/cm² to 2 μg/cm², preferably 0.2 μg/cm² to 1 μg/cm², and more preferably 0.2 μg/cm² to 0.7 μg/cm².

The total content of the antibody (B) or an antigen-binding fragment thereof and the antibody (C) or an antigen-binding fragment thereof contained in the detection part (4) is usually 0.2 μg to 2 μg, preferably 0.4 μg to 1.4 μg, and more preferably 0.6 μg to 1.2 μg.

Further, the total content of the antibody (B) or an antigen-binding fragment thereof and the antibody (C) or an antigen-binding fragment thereof per unit area of the detection part (4) is usually 0.3 μg/cm² to 4 μg/cm², preferably 0.4 μg/cm² to 2 μg/cm², and more preferably 0.4 μg/cm² to 1.4 μg/cm².

The content ratio of the antibody (B) or an antigen-binding fragment thereof to the antibody (C) or an antigen-binding fragment thereof contained in the detection part (4) is preferably 1:2 to 2:1, and more preferably 2:1 in mass ratio.

In addition, the detection part (4) preferably contains the antibody (E) or an antigen-binding fragment thereof. When the antibody (E) or an antigen-binding fragment thereof is contained, the content of the antibody (E) or an antigen-binding fragment thereof in the labeling substance retaining part is usually 0.1 μg to 1 μg, preferably 0.2 μg to 0.7 μg, and more preferably 0.3 μg to 0.6 μg. Further, the content of the antibody (E) or an antigen-binding fragment thereof per unit area of the detection part (4) is usually 0.15 μg/cm² to 4 μg/cm², preferably 0.2 μg/cm² to 3 μg/cm², and more preferably 0.2 μg/cm² to 2.5 μg/cm².

Further, the content ratio of the total content of the antibody (B) or an antigen-binding fragment thereof and the antibody (C) or an antigen-binding fragment thereof in the labeling substance retaining part to the antibody (E) or an antigen-binding fragment thereof therein is preferably 1:2 to 2:1 in mass ratio.

The absorption part (5) is placed at the end of the chromatographic medium part (3) in order to absorb a liquid such as a specimen or a developing solution that has passed through the detection part (4). In the present invention, as the absorption part (5), for example, glass fiber, pulp, cellulose fiber, or the like, or a material in which a polymer such as an acrylic acid polymer and a hydrophilic agent having an ethylene oxide group or the like are incorporated in a non-woven fabric thereof is used, and glass fiber is particularly preferred.

The backing sheet (6) is an arbitrary base material. By applying an adhesive to one surface or sticking an adhesive tape to one surface, the surface has adhesiveness, and on the adhesive surface, part or all of the sample loading part (1), the labeling substance retaining part (2), the chromatographic medium part (3), the detection part (4), and the absorption part (5) are provided in close contact with one another. The backing sheet (6) is not particularly limited as the base material as long as it becomes impermeable to a sample solution and impermeable to moisture by the adhesive.

The immunochromatographic analysis device according to the present invention is usually subjected to a drying treatment before being made into a product. The drying temperature is, for example, 20° C. to 50° C., and the drying time is 0.5 hours to 1 hour.

<Immunochromatographic Analysis Kit>

The immunochromatographic analysis kit according to the present invention includes the immunochromatographic analysis device and a specimen diluent for diluting and developing the specimen.

In the immunochromatographic analysis kit according to the present invention, the specimen diluent can also be used as a developing solution, but water is usually used as a solvent, and a buffer solution, a salt such as casein sodium, a surfactant such as a nonionic surfactant, a cationic surfactant, or an anionic surfactant, a polymer compound such as polyvinylpyrrolidone (PVP), a polyanion, a nitrogen atom-containing vinyl-based water-soluble polymer, an antibacterial agent, a chelating agent, or the like can be incorporated therein. Among these, one type or two or more types may be incorporated.

As the surfactant, a surfactant having an HLB value of 13 to 17 can be preferably used. Examples thereof include Triton X-100 (trade name, polyethylene glycol mono-p-isooctylphenyl ether, HLB: 13.7), Tween 20 (trade name, polyoxyethylene sorbitan monolaurate, HLB: 16.7), Tween 80 (trade name, polyoxyethylene sorbitan monooleate, HLB: 15.0), Brij 35 (polyoxyethylene glycol dodecyl ether, HLB: 13.7), and the like, and one type or two or more types may be added. By incorporating a surfactant having an HLB value of 13 to 17 in the sample loading part, an antigen-antibody non-specific reaction in the labeling substance retaining part and the detection part can be suppressed, and the detection sensitivity to the detection target in the detection part can be increased.

The content of the nonionic surfactant in the specimen diluent is usually 0.5% to 5%, preferably 0.5% to 3%, and more preferably 0.5% to 2%.

Further, examples of the nitrogen atom-containing vinyl-based water-soluble polymer include polyvinylpyrrolidone (PVP), and the like. By incorporating a nitrogen atom-containing vinyl-based water-soluble polymer in the specimen diluent, it is possible to enhance the signal by arbitrarily aggregating the insoluble carrier that has reacted with the antigen, and therefore, the detection sensitivity to the detection target in the detection part can be increased.

The content of the nitrogen atom-containing vinyl-based water-soluble polymer in the specimen diluent is usually 0.1% to 0.8%, preferably 0.2% to 0.6%, and more preferably 0.2% to 0.5%.

The specimen diluent is mixed with a specimen sample in advance, and the obtained specimen-containing liquid can be loaded in the sample loading part to cause development, or a specimen sample is loaded in the sample loading part in advance, and thereafter, the specimen diluent may be loaded in the sample loading part to cause development.

<Immunochromatographic Analysis Method>

The immunochromatographic analysis method according to the present invention includes the following steps (1) to (4), and is a method for detecting chikungunya virus contained in a specimen using the immunochromatographic analysis kit:

(1) a step of loading a specimen-containing liquid obtained by diluting a specimen with a specimen diluent in the sample loading part;

(2) a step of allowing the antibody (A) or an antigen-binding fragment thereof held by the labeling substance retaining part to recognize the chikungunya virus;

(3) a step of developing the specimen and the antibody (A) or an antigen-binding fragment thereof in the chromatographic medium part as a mobile phase; and

(4) a step of detecting the chikungunya virus in the developed mobile phase with the antibody (B) or an antigen-binding fragment thereof and the antibody (C) or an antigen-binding fragment thereof contained in the detection part.

The respective steps will be described below.

(1) Step of loading a specimen-containing liquid obtained by diluting a specimen with a specimen diluent in the sample loading part

In the step (1), in the first place, it is preferred that a specimen-containing liquid is prepared by adjusting or diluting a specimen with a specimen diluent to such a concentration that the specimen smoothly migrates in the device without decreasing the measurement accuracy. As the specimen diluent, a diluent described above can be used. In the second place, as the sample, the specimen-containing liquid is loaded on the sample loading part (1) in a predetermined amount (usually 0.1 mL to 2 mL). When the sample is loaded on the sample loading part (1), the sample starts to migrate in the sample loading part (1). As the specimen to be used in the present invention, a material described above can be used.

(2) Step of allowing the antibody (A) or an antigen-binding fragment thereof held by the labeling substance retaining part to recognize the chikungunya virus

The step (2) is a step in which the sample loaded in the sample loading part in the step (1) is allowed to migrate to the labeling substance retaining part (2), and the antibody (A) or an antigen-binding fragment thereof, which is held in the labeling substance retaining part, and to which the labeling substance is bound, is allowed to recognize chikungunya virus in the specimen. As the labeling substance, a substance described above can be used.

(3) Step of developing the specimen and the antibody (A) or an antigen-binding fragment thereof in the chromatographic medium part as a mobile phase

The step (3) is a step in which after the chikungunya virus in the specimen is recognized by the antibody (A) or an antigen-binding fragment thereof to which the labeling substance is bound in the labeling substance retaining part in the step (2), the specimen and the antibody (A) or an antigen-binding fragment thereof are allowed to pass on the chromatographic medium part as a mobile phase.

(4) Step of detecting the chikungunya virus in the developed mobile phase with the antibody (B) or an antigen-binding fragment thereof and the antibody (C) or an antigen-binding fragment thereof contained in the detection part

The step (4) is a step in which the chikungunya virus in the specimen that has passed on the chromatographic medium part as the mobile phase specifically reacts and binds so as to be interposed between the antibody (B) or an antigen-binding fragment thereof and the antibody (C) or an antigen-binding fragment thereof fixed to the detection part and the antibody (A) or an antigen-binding fragment thereof to which the labeling substance is bound in the step (2) in a sandwich-like form by an antigen-antibody specific binding reaction, so that the detection part is colored.

When chikungunya virus that is the detection target is not present, a labeling reagent dissolved in an aqueous component of the sample does not cause a specific binding reaction even if it passes through the detection part on the chromatographic medium part, and therefore, the detection part is not colored.

Finally, the aqueous component of the specimen-containing liquid migrates to the absorption part (5).

### EXAMPLES

Hereinafter, the present invention will be further described by way of Examples, however, the present invention is not limited to the following examples.

(1) Preparation of Antibodies

(1-1) 13H11, 3D11, and 15B2 Monoclonal Antibodies

As the antigens, cultured cells infected with ECSA genotype CHIKV and Sendai virus that expresses the 6K-E1 protein of Asian genotype CHIKV were used. Specifically, first immunization was carried out in mice (Balb/c) at 4 to 6 weeks of age with a mixture of the cultured cells and CFA (complete Freund's adjuvant). Two weeks after the first immunization, second immunization was carried out in the mice with a mixture of the cultured cells and IFA (incomplete Freund's adjuvant). Two weeks after the second immunization, third immunization was carried out in the mice with a mixture of the virus and IFA. On day 3 after the third immunization, B cells were prepared from the mice, and hybridomas were prepared from the B cells by a conventional method. With respect to the obtained hybridomas, anti-CHIKV antibody-producing hybridomas were screened using the culture solution.

As a result, three types of hybridomas (13H11, 3D11, and 15B2 strains) were isolated. As the anti-CHIKV antibodies produced from the hybridomas, three types of monoclonal antibodies (13H11, 3D11, and 15B2) were obtained. The isotypes of 13H11, 3D11, and 15B2 were IgG1κ, IgG2aκ, and IgG2bκ, respectively.

13H11 corresponds to the antibody (A), 3D11 corresponds to the antibody (B), and 15B2 corresponds to the antibody (C).

The amino acid sequences of 13H11, 3D11, and 15B2 that are the anti-CHIKV antibodies were determined. These results are shown below. Note that the light chains of 13H11, 3D11, and 15B2 all have the same amino acid sequence. Further, in each amino acid sequence, the amino acid sequences indicated by underlines correspond to the amino acid sequences of CDR1, CDR2, and CDR3 from the N-terminus to the C-terminus, respectively. In addition, in each amino acid sequence, the amino acid sequence enclosed in parentheses corresponds to the amino acid sequence of the variable region.

Heavy Chain of 13H11
(SEQ ID NO: 17)
[QVQLQQSGPELVKPGASVKISCKASGYAFSTSWMNWVKQRPGQGLEWIG
RIYPGDGDTNYNGKFKGKATLTADKSSSTAYMQLSSLTSVDSAVYFCARS
NDGYYVGYWGQGTTLTVSS]ASTTPPSVYPLAPGSAAQTNSMVTLGCLVK
GYFPEPVTVTWNSGSLSSGVHTFPAVLQSDLYTLSSVTVPSSTWPSETVT
CNFAHPASSTKVDKKIVPRDCGCKPCICTVPEVSSYFIFPPKPKDVLTIT
LTPKVTCVVVDISKDDPEVQFSWFVDDVEVHTAQTQPREEQFNSTFRSVS
ELPIMHQDWLNGKEFKCRVNSAAFPAPIEKTISKTKGRPKAPQVYTIPPP
KEQMAKDKVSLTCMITDFFPEDITVEWQWNGQPAENYKTQPIMDTDGSYF
VYSKLNVQKSNWEAGNTFTCSVLHEGLHNHHTEKSLSHSPGK
Heavy Chain of 3D11
(SEQ ID NO: 19)
[QVQLQQSGAELVARGASVKLSCKASGYTFTSYWMQWVKQRPGQGLEWIG
AIYPGDGDTRYTQKFKGKATLTADKSSSTAYMQLSSLASEDSAVYYCARS
YDPFDYWGQGTTLTVSS]AKTTAPSVYPLAPVCGDTTGSSVLTGCLVKGY
FPEPVTLTWNSGLSSGVHTFPAVLQSDLYTLSSSVTVTSSTWPSQSITCN
VAHPASSTKVDKKIEPRGPTIKPCPPCKCPAPNLLGGPSVFIFPPKIKDV
LMISLSPIVTCVVVDVSEDDPDVQISWFVNNVEVHTAQTQTHREDYNSTL
RVVSALPIQHQDWMSGKEFKCKVNNKDLPAPIERTISKPKGSVRAPQVYV
LPPPEEEMTKKQVTLTCMVDTFMPEDIYVEWTNNGKTELNYKNTEPVLDS
DGSYFMYSKLRVEKKNWVERNSYSCSVVHEGLHNHHTTKSFSRTPGK
Heavy Chain of 15B2
(SEQ ID NO: 20)
[QVQLQQSGAELVKPGASVKLSCKASGYTFTSYYMYWVKQRPGQGLEWIG
EINPSNGGTNFNEKFKNKATLTVDKSSNTAYMQLNSLTSEDSAVYYCTRG
YYGNPFFAYWGQGTLVTVSA]AKTTPPSVYPLALGCGDTTGSSVTLGCLV
KGYFPESVTVTWNSGSLSSSVHTFPALLQSGLYTMSSSVTVPSSTWPSQT
VTCSVAHPASSTTVDKKLEPSGPISTINPCPPCKECHKCPAPNLEGGPSV
FIFPPNIKDVLMISLTPKVTCVVVDVSEDDPDVQISWFVNNVEVHTAQTQ
THREDYNSTIRVVSTLPIQHQDWMSGKEFKCKVNNKDLPSPIERTISKIK
GLVRAPQVYILPPPAEQLSRKDVSLTCLVVGFNPGDISVEWTSNGHTEEN
YKDTAPVLDSDGSYFIYSKLNMKTSKWEKTDSFSCNVRHEGLKNYYLKKT
ISRSPGK
Light Chain of 13H11, 3D11, and 15B2
(SEQ ID NO: 18)
[NIVLTQSPKSMSMSVGERVTLTCKASENVVTYVSWYQQKPEQSPKLLIY
GASNRYTGVPDRFTGSGSATDFTLTISSVQAEDLADYHCGQGYSYPYTFG
GGTKLEI]KRADAAPTVSIFPPSSEQLTSGGASVVCFLNNFYPKDINVKW
KIDGSERQNGVLNSWTDQDSKDSTYSMSSTLTLTKDEYERHNSYTCEATH
KTSTSPIVKSFNRNEC

(1-2) CK-(d) and CK-(e) Monoclonal Antibodies

B7 cells were infected with CHIKV and incubated until a wide range of cytopathic effect was observed. The infected cells were inactivated with phosphate buffered saline (PBS) containing 4% formaldehyde overnight at 4° C., and then collected, washed 3 times with PBS, and stored at −80° C. prior to injection. Female BALB/c mice (National Laboratory Animal Center, Mahidol University, Bangkok, Thailand) at 5 weeks of age were intraperitoneally immunized with the inactivated CHIKV-infected B7 cells (2.5×10⁶ infected cells/mouse) in complete Freund's adjuvant (Sigma Aldrich, Saint Louis, Mo.). In addition, booster immunization was carried out 3 times every 3 weeks with the CHIKV-infected cells without adjuvant. Three days after the final booster immunization, the spleen was excised, the spleen cells were obtained and fused with mouse myeloma PAI cells using polyethylene glycol #1500 (Roche diagnostics, Mannheim, Germany) to prepare hybridomas. The hybridomas were cultured in RPMI 1640 supplemented with 15% FBS, hypoxanthine-aminopterin-thymidine (GIBCO, Grand Island, N.Y.), and 3% BM-Condimed H1 supplement (Roche diagnostics). Antibodies secreted by the hybridomas were screened by an indirect immunofluorescence assay (IFA) using CHIKV-infected Vero cells.

As a result, two types of hybridomas (CK-(d) and CK-(e) strains) were isolated. Two types of monoclonal antibodies (CK-(d) and CK-(e)) were obtained as the anti-CHIKV antibodies produced from these hybridomas. The isotypes of CK-(d) and CK-(e) were IgG1 and IgG2a, respectively.

CK-(d) corresponds to the antibody (D), and CK-(e) corresponds to the antibody (E).

The amino acid sequences of CK-(d) and CK-(e) that are the anti-CHIKV antibodies were determined. These results are shown below. Note that the light chains of CK-(d) and CK-(e) both have the same amino acid sequence as those of the above-mentioned 13H11, 3D11, and 15B2. Further, in each amino acid sequence, the amino acid sequences indicated by underlines correspond to the amino acid sequences of CDR1, CDR2, and CDR3 from the N-terminus to the C-terminus, respectively. In addition, in each amino acid sequence, the amino acid sequence enclosed in parentheses corresponds to the amino acid sequence of the variable region.

Heavy Chain of CK-(d)
(SEQ ID NO: 29)
[ESGGGLVKLGGSLKLSCAASGFTFSTYYMSWVRQTPEKRLELVAAINSN
GGSTYYPDTVKGRFTISRDNAKNTLYLQMSSLKSEDTALYYCARHELVGG
WFVYWGQGTLVTVSA]AKTTPPSVYPLAPGSAAQTNSMVTLGCLVKGYFP
EPVTVTWNSGSLSSGVHTFPAVLQSDLYTLSSSVTVPSSTWPSETVTCNV
AHPASSTKVDKKIVPRDCGCKPCICTVPEVSSVFIFPPKPKDVLTITLTP
PKVTCVVVDISKDDPEVQFSWFVDDVEVHTAQTQPREEQFNSTFRSVSEL
PIMHQDWLNGKEFKCRVNSAAFPAPIEKTISKTKGRPKAPQVYTIPPPKE
QMAKDKVSLTCMITDFFPEDITVEWQWNGQPAENYKNTQPIMDTDGSYFV
YSKLNVQKSNWEAGNTFTCSVLHEGLHNHHTEKALAHSPGK
Heavy Chain of CK-(e)
(SEQ ID NO: 30)
[QVQLQQSGAELVRPGTSVKMSCKAAGYTFTNYWIGWIKQRPGHGLEWIG
DVYPGGGSTYYNEKFKAKATLTADTSSSTAYMQLSRLTSEDSAIYYCSRV
TSTTGWFYDVWGAGTTVTVSS]AKTTAPSVYPLALVCGDTTGSSVTLGCL
VKGYFPEPVTLTWNSGSLSSGVHTFPAVLQSDLYTLSSSVTVTSSTWPSQ
SITCNVAHPASSTKVDKKIEPRGPTIKPCPPCKCPAPNLLGGPSVFIFPP
KIKDVLMISLSPIVTCVVVDVSEDDPDVQISWFVNNVEVHTAQTQTHRED
YNSTLRVVSALPIQHQDWMSGKEFKCKVNNKDLPAPIERTISKPKGSVRA
PQVYVLPPPEEEMTKKQVTLTCMVTDFMPEDIYVEWTNNGKTELNYKNTE
PVLDSDGSYFMYSKLRVEKKNWVERNSYSCSVVHEGLHNHHTTKSFSRTP
GK

(2) Preparation of Immunochromatographic Analysis Device

### Example 1

An immunochromatographic analysis kit composed of a specimen diluent and an immunochromatographic analysis device including a sample loading part (1), a labeling substance retaining part (2), a chromatographic medium part (3) having a detection part (4), and an absorption part (5) was prepared.

1. Preparation of Sample Loading Part

As a sample loading part, a non-woven fabric composed of glass fiber (manufactured by Millipore, Inc., 300 mm×30 mm) was used.

2. Preparation of Labeling Substance Retaining Part

The 13H11 monoclonal antibody was diluted to a concentration of 0.05 mg/mL with a phosphate buffer solution (pH 7.4), whereby an antibody solution was obtained. Then, 0.1 mL of the antibody solution was added to 0.5 mL of a colloidal gold suspension (manufactured by Tanaka Kikinzoku Kogyo K.K., LC 40 nm), and the resulting mixture was left to stand at room temperature for 10 minutes.

Subsequently, 0.1 mL of a phosphate buffer solution (pH 7.4) containing 1 mass % bovine serum albumin (BSA) was added thereto, and the resulting mixture was left to stand at room temperature for an additional 10 minutes. Thereafter, the mixture was thoroughly stirred, and then centrifuged at 8000×g for 15 minutes. After removing the supernatant, 0.1 mL of a phosphate buffer solution (pH 7.4) containing 1 mass % BSA was added thereto. According to the above-mentioned procedure, a labeling substance solution was prepared.

A solution obtained by adding 300 μL of a 10 mass % trehalose aqueous solution and 1.8 mL of distilled water to 300 μL of the labeling substance solution prepared above was added uniformly to a 16 mm×300 mm glass fiber pad (manufactured by Millipore, Inc.), followed by drying in a vacuum dryer, whereby a labeling substance retaining part was prepared.

The content of the 13H11 monoclonal antibody in the labeling substance retaining part was 0.1 μg (0.18 μg/cm²).

3. Preparation of Chromatographic Medium Part and Detection Part

As a membrane, a sheet composed of nitrocellulose (manufactured by Millipore, Inc., trade name: HF 120, 300 mm×25 mm) was used.

Subsequently, 150 μL of an antibody solution obtained by adjusting each of the 3D11 monoclonal antibody to a concentration of 0.3 mg/mL and the 15B2 monoclonal antibody to a concentration of 0.6 mg/mL with a phosphate buffer solution (pH 7.4) containing 5 mass % isopropyl alcohol was applied in a line shape with a width of 1 mm in an amount of 1 μL/mm (25 sL per sheet) to the detection part on the dried membrane using a dispenser for immunochromatography “XYZ3050” (manufactured by BioDot, Inc.).

Further, in order to confirm the presence or absence of development of a gold nanoparticle labeling reagent or the developing rate, on the downstream of the detection part, a solution obtained by diluting a goat-derived antiserum having affinity in a wide range for the gold nanoparticle labeling substance with a phosphate buffer solution (pH 7.4) was applied to a control region (control line). Thereafter, the solution was dried at 50° C. for 30 minutes, and then dried overnight at room temperature, whereby a chromatographic medium part and a detection part were prepared.

In the detection part, the content of the 3D11 monoclonal antibody was 0.14 μg (0.25 μg/cm²), and the content of the 15B2 monoclonal antibody was 0.14 μg (0.25 μg/cm²).

4. Preparation of Immunochromatographic Analysis Device

Subsequently, to a base material composed of a backing sheet (manufactured by Kuramoto Sangyo Co.), the sample loading part, the labeling substance retaining part, the chromatographic medium part having the detection part, and a non-woven cloth made of glass fiber as an absorption part for absorbing the developed sample and labeling substance were sequentially bonded. Then, the resulting material was cut to a width of 3.5 mm by a cutting machine, whereby an immunochromatographic analysis device was prepared. Note that the length in the sample developing direction of the labeling substance retaining part was set to 16 mm.

5. Preparation of Specimen Diluent

A 50 mM HEPES buffer solution (pH 7.5) containing a 1 mass % nonionic surfactant (a 1:1 mixture of NP-40 manufactured by Nacalai Tesque, Inc. and Nonidet MN-811 manufactured by NOF Corporation) was prepared and used as a specimen diluent for performing a dilution treatment of a specimen.

### Example 2

An immunochromatographic analysis kit of Example 2 was prepared in the same manner as in Example 1 except that in Example 1, the CK-(d) monoclonal antibody was used in addition to the 13H11 monoclonal antibody in the labeling substance retaining part, the CK-(e) monoclonal antibody was used in addition to the 3D11 monoclonal antibody and the 15B2 monoclonal antibody in the detection part, and the content of each antibody in the labeling substance retaining part and the detection part was set as follows.

In the labeling substance retaining part, the content of the 13H11 monoclonal antibody was 0.05 μg (0.09 μg/cm²) and the content of the CK-(d) monoclonal antibody was 0.05 μg (0.09 μg/cm²), and in the detection part, the content of the 3D1 monoclonal antibody was 0.06 μg (0.69 μg/cm²), the content of the 15B2 monoclonal antibody was 0.1 μg (1.1 μg/cm²), and the content of the CK-(e) monoclonal antibody was 0.2 μg (2.29 μg/cm²).

### Example 3

An immunochromatographic analysis kit of Example 3 was prepared in the same manner as in Example 2 except that in Example 2, in the labeling substance retaining part, the content of the 13H11 monoclonal antibody was set to 0.03 μg (0.05 μg/cm²) and the content of the CK-(d) monoclonal antibody was set to 0.06 μg (0.1 μg/cm²) so that the mass ratio of the content of the 13H11 monoclonal antibody to the content of the CK-(d) monoclonal antibody was 1:2.

### Comparative Example 1

An immunochromatographic analysis kit of Comparative Example 1 was prepared in the same manner as in Example 1 except that in Example 1, only the CK-(d) monoclonal antibody was used in place of the 13H11 monoclonal antibody, only the CK-(e) monoclonal antibody was used in place of the 3D1 monoclonal antibody and the 15B2 monoclonal antibody, and the content of the CK-(d) monoclonal antibody in the labeling substance retaining part was set to 0.1 μg (0.18 μg/cm²), and the content of the CK-(e) monoclonal antibody in the detection part was set to 0.29 μg (3.3 μg/cm²).

The combinations of antibodies contained in the labeling substance retaining part and the detection part in Examples 1 to 3 and Comparative Example 1 are summarized in the following Table 1.

TABLE 1

| | Labeling substance retaining part | | Detection part | | |
|---|---|---|---|---|---|
| Antibody | 13H11 | CK-(d) | 3D11 | 15B2 | CK-(e) |
| Example 1 | ✓ | | ✓ | ✓ | |
| Example 2 | ✓ | ✓ | ✓ | ✓ | ✓ |
| Example 3 | ✓ | ✓ | ✓ | ✓ | ✓ |
| Comparative | | ✓ | | | ✓ |
| Example 1 | | | | | |

### Example 1

### Test Example 1

In this test, in the immunochromatographic analysis kits of Examples 1 to 3 and Comparative Example 1, it was examined whether a difference in reactivity occurred among chikungunya viruses with different genotypes. Specifically, by using the immunochromatographic analysis kits of Examples 1 to 3 and Comparative Example 1 prepared above, measurement was carried out using the following specimens.

As the chikungunya viruses of the specimens, ECSA genotype (autologous culture), Asian genotype (autologous culture), and WA genotype (autologous culture) were used. As for the WA genotype (autologous culture), a pseudotype virus in which the envelope was changed to one derived from chikungunya virus was prepared and used for HIV lentivector.

As for the ECSA genotype and the Asian genotype, specimen-containing liquids were prepared by diluting each virus to 1×10⁴ pfu/mL, 1×10⁵ pfu/mL, or 1×10⁶ pfu/mL with the specimen diluent, and used as specimen samples. Further, as a negative control specimen, a specimen diluent containing no virus was used.

In addition, as for the WA genotype (autologous culture), specimen-containing liquids were prepared by diluting each virus to 6 ng/mL, 30 ng/mL, and 150 ng/mL with the specimen diluent, and used as specimen samples.

Further, as a negative control specimen, a specimen diluent containing no virus was used.

90 μL of each of the specimen-containing liquids or the negative specimen prepared above was loaded in the sample loading part of the immunochromatographic analysis device and developed, and after 15 minutes, the developed color intensity of the detection part was measured using an immunochromatographic reader and evaluated as follows.

−: 0 mAbs or more and less than 10 mAbs (determined to be negative)

±: 10 mAbs or more and less than 15 mAbs (determined to be positive)

+: 15 mAbs or more and less than 150 mAbs (determined to be positive)

++: 150 mAbs or more and less than 300 mAbs (determined to be positive)

+++: 300 mAbs or more (determined to be positive)

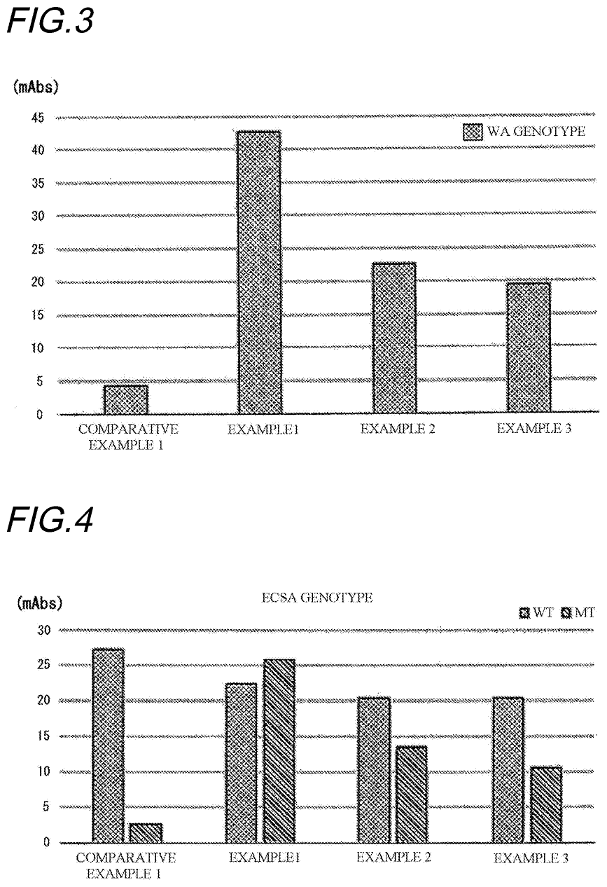

The results are shown in Tables 2 to 5. In addition, as for the ECSA genotype and the Asian genotype, a graph summarizing the results for each genotype when the virus concentration was set to 1×10⁴ pfu/mL is shown in FIG. 2, and as for the WA genotype, a graph summarizing the results when the virus concentration was set to 6 ng/mL is shown in FIG. 3.

TABLE 2

| | | | | | | (mAbs) |
|---|---|---|---|---|---|---|
| ECSA genotype | 1 × 10⁴ pfu/mL | | 1 × 10⁵ pfu/mL | | 1 × 10⁶ pfu/mL | |
| Comparative | 28.3 | + | 235.4 | ++ | 663.2 | +++ |
| Example 1 | | | | | | |
| Example 1 | 13.6 | ± | 49.5 | + | 242.4 | ++ |
| Example 2 | 26.6 | + | 125.7 | + | 215.4 | ++ |
| Example 3 | 22.4 | + | 176.6 | ++ | 645.9 | +++ |

TABLE 3

| | | | | | | (mAbs) |
|---|---|---|---|---|---|---|
| Asian genotype | 1 × 10⁴ pfu/mL | | 1 × 10⁵ pfu/mL | | 1 × 10⁶ pfu/mL | |
| Comparative | 3.4 | − | 83 | + | 629.3 | +++ |
| Example 1 | | | | | | |
| Example 1 | 29.7 | + | 149 | ++ | 290.7 | ++ |
| Example 2 | 18.1 | + | 185.4 | ++ | 534.2 | +++ |
| Example 3 | 18.4 | + | 161.7 | ++ | 610.3 | +++ |

TABLE 4

| | | | | | | (mAbs) |
|---|---|---|---|---|---|---|
| WA genotype | 6 ng/mL | | 30 ng/mL | | 150 ng/mL | |
| Comparative | 4.4 | − | 15.1 | + | 151.5 | ++ |
| Example 1 | | | | | | |
| Example 1 | 42.7 | + | 163.6 | ++ | 390.6 | +++ |
| Example 2 | 22.5 | + | 96 | + | 258.5 | ++ |
| Example 3 | 19.4 | + | 50.8 | + | 281.5 | ++ |

TABLE 5
(mAbs)

| Negative | | |
|---|---|---|
| specimen | After 15 min | |
| Comparative | 1.8 | — |
| Example 1 | | |
| Example 1 | 3.9 | — |
| Example 2 | 3.7 | — |
| Example 3 | 7.8 | — |

From the results of Test Example 1, it was found that the ECSA genotype, Asian genotype, and WA genotype chikungunya viruses can be detected in any of the immunochromatographic analysis kits of Examples 1 to 3. Further, when the developed color intensities of Examples 2 and 3 were compared with those of Comparative Example 1, it was found that the developed color intensity for the ECSA genotype is higher than that of Example 1, and the difference due to genotype is further eliminated. On the other hand, in the immunochromatographic analysis kit of Comparative Example 1, it was determined that the result for the Asian genotype was negative when the virus concentration was 1×10⁴.

Note that as can be seen also from the results of the negative specimen, no non-specific reaction (false positive reaction) was observed in any of the immunochromatographic analysis kits.

### Test Example 2

In this test, in the immunochromatographic analysis kits of Examples 1 to 3 and Comparative Example 1, a difference in reactivity depending on the presence or absence of mutation in chikungunya virus were examined. Specifically, by using the immunochromatographic analysis kits of Examples 1 to 3 and Comparative Example 1 prepared above, measurement was carried out using the following specimens.

As the chikungunya viruses of the specimens, autologous cultures of ECSA wild type (WT) and mutant type (MT), and Asian wild type (WT) and mutant type (MT) were used, and specimen-containing liquids were prepared by diluting each virus to 6 ng/mL, 30 ng/mL, and 150 ng/mL with the specimen diluent, and used as specimen samples.

The CSA mutant type is one obtained by substituting glutamic acid at position 350 in the amino acid sequence of the ECSA wild type E1 protein with aspartic acid, and the Asian mutant type is one obtained by substituting aspartic acid at position 350 in the amino acid sequence of the Asian wild type E1 protein with glutamic acid.

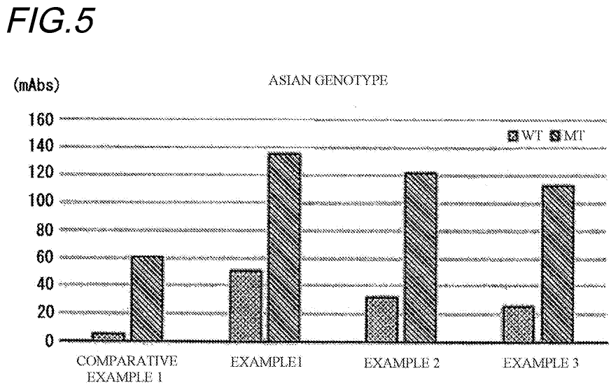

90 μL of each of the specimen-containing liquids prepared above was loaded in the sample loading part of the immunochromatographic analysis device and developed, and after 15 minutes, the developed color intensity of the detection part was measured using an immunochromatographic reader and evaluated in the same manner as in Test Example 1. The results are shown in Tables 6 to 9. In addition, graphs summarizing the results for each genotype when the virus concentration was set to 6 ng/mL are shown in FIG. 4 and FIG. 5.

TABLE 6
(mAbs)

| ECSA genotype WT | 6 ng/mL | | 30 ng/mL | | 150 ng/mL | |
|---|---|---|---|---|---|---|
| Comparative | 27.2 | + | 107.4 | + | 325.9 | +++ |
| Example 1 | | | | | | |
| Example 1 | 22.4 | + | 53.5 | + | 167.9 | ++ |
| Example 2 | 20.4 | + | 73.9 | + | 225.9 | ++ |
| Example 3 | 20.3 | + | 85 | + | 306.2 | +++ |

TABLE 7
(mAbs)

| ECSA genotype MT | 6 ng/mL | | 30 ng/mL | | 150 ng/mL | |
|---|---|---|---|---|---|---|
| Comparative | 2.7 | − | 17.3 | + | 112.4 | + |
| Example 1 | | | | | | |
| Example 1 | 25.9 | + | 68.5 | + | 200.5 | ++ |
| Example 2 | 13.5 | ± | 45.7 | + | 193.9 | ++ |
| Example 3 | 10.5 | ± | 39.4 | + | 175.1 | ++ |

TABLE 8
(mAbs)

| Asian genotype WT | 6 ng/mL | | 30 ng/mL | | 150 ng/mL | |
|---|---|---|---|---|---|---|
| Comparative | 5.7 | − | 14 | ± | 130.8 | + |
| Example 1 | | | | | | |
| Example 1 | 50.8 | + | 164.4 | ++ | 385.7 | +++ |
| Example 2 | 32.4 | + | 128.9 | + | 282.1 | ++ |
| Example 3 | 25.6 | + | 101.6 | + | 319.6 | +++ |

TABLE 9
(mAbs)

| Asian genotype MT | 6 ng/mL | | 30 ng/mL | | 150 ng/mL | |
|---|---|---|---|---|---|---|
| Comparative | 60.6 | + | 253.4 | ++ | 518.7 | +++ |
| Example 1 | | | | | | |
| Example 1 | 135.9 | + | 366 | +++ | 531.2 | +++ |
| Example 2 | 121.7 | + | 343.8 | +++ | 542.4 | +++ |
| Example 3 | 113.1 | + | 356.5 | +++ | 508.2 | +++ |

As can be seen from the results of Tables 6 and 7, with respect to the ECSA genotype, in the immunochromatographic analysis kits of Examples 1 to 3, a positive reaction was obtained for both the wild type and the mutant type when the virus concentration was 6 ng/mL. On the other hand, in the immunochromatographic analysis kit of Comparative Example 1, the result for the mutant type was determined to be negative.

Further, as can be seen from the results of Tables 8 and 9, with respect to the Asian genotype, in the immunochromatographic analysis kits of Examples 1 to 3, a positive reaction was obtained for both the wild type and the mutant type when the virus concentration was 6 ng/mL. On the other hand, in the immunochromatographic analysis kit of Comparative Example 1, the result for the wild type was determined to be negative.

From the results of Test Example 2, it was found that in the immunochromatographic analysis kits of Examples 1 to 3, the difference in reactivity between the wild type and the mutant type is small as compared with the immunochromatographic analysis kit of Comparative Example 1.

### Test Example 3

In this test, in the immunochromatographic analysis kits of Examples 1 to 3 and Comparative Example 1, cross-reactivity with a virus other than chikungunya virus was examined. Specifically, the immunochromatographic analysis kits of Examples 1 to 3 and Comparative Example 1 prepared above were used, as the specimen, Sindbis virus (SINV, autologous culture) that belongs to the genus Alphavirus of the family Togaviridae in the same manner as chikungunya virus was used, and specimen-containing liquids were prepared by diluting the virus to 1×10⁵ pfu/mL, 1×10⁶ pfu/mL, or 1×10⁷ pfu/mL with the specimen diluent, and used as specimen samples.

90 μL of each of the specimen-containing liquids prepared above was loaded in the sample loading part of each of the immunochromatographic analysis devices of Examples 2 and 3 and Comparative Example 1 and developed, and after 15 minutes, the developed color intensity of the detection part was measured using an immunochromatographic reader. The results are shown in Table 10 (ND indicates not measurable).

TABLE 10

| | | | | | | (mAbs) |
|---|---|---|---|---|---|---|
| SINV | 1 × 10⁵ pfu/mL | | 1 × 10⁶ pfu/mL | | 1 × 10⁷ pfu/mL | |
| Comparative | 0 | − | 2.1 | − | 43.9 | + |
| Example 1 | | | | | | |
| Example 2 | ND | | 0 | − | 7.8 | − |
| Example 3 | 0 | − | 0 | − | 5.5 | − |

As can be seen from the above results, in the immunochromatographic analysis kits of Examples 2 and 3, no cross-reactivity with Sindbis virus was observed. On the other hand, in the immunochromatographic analysis kit of Comparative Example 1, cross-reactivity with Sindbis virus was observed when the virus concentration was 1-10 pfu/mL.

While the present invention has been described in detail with reference to specific embodiments, it is apparent to those skilled in the art that various changes and modifications can be made without departing from the spirit and scope of the present invention. The present application is based on Japanese Patent Application (Japanese Patent Application No. 2018-022527) filed on Feb. 9, 2018, the entire contents of which are incorporated herein by reference.

### REFERENCE SIGNS LIST

1. sample loading part
2. labeling substance retaining part
3. chromatographic medium part
4. detection part
5. absorption part
6. backing sheet

1. An immunochromatographic analysis device for detecting chikungunya virus, comprising a sample loading part, a labeling substance retaining part, a chromatographic medium part having a detection part, and an absorption part, wherein
the labeling substance retaining part contains the following antibody (A) or an antigen-binding fragment thereof, and
the detection part contains the following antibody (B) or an antigen-binding fragment thereof, and the following antibody (C) or an antigen-binding fragment thereof,
(A) an antibody against the chikungunya virus, including the following heavy chain variable region (1) and the following light chain variable region (2),
(1) a heavy chain variable region including a heavy chain complementarity determining region (CDRH) 1, CDRH2, and CDRH3,
CDRH1 being a polypeptide containing the following amino acid sequence (H1-A),
CDRH2 being a polypeptide containing the following amino acid sequence (H2-A), and
CDRH3 being a polypeptide containing the following amino acid sequence (H3-A),
(H1-A) the following amino acid sequence (H1-A1), (H1-A2), or (H1-A3),
(H1-A1) an amino acid sequence of SEQ ID NO: 1,
(H1-A2) an amino acid sequence having 80% or more identity with the amino acid sequence of SEQ ID NO: 1,
(H1-A3) an amino acid sequence in which one or several amino acids are deleted, substituted, inserted, and/or added in the amino acid sequence of SEQ ID NO: 1,
(H2-A) the following amino acid sequence (H2-A1), (H2-A2), or (H2-A3),
(H2-A1) an amino acid sequence of SEQ ID NO: 2,
(H2-A2) an amino acid sequence having 80% or more identity with the amino acid sequence of SEQ ID NO: 2,
(H2-A3) an amino acid sequence in which one or several amino acids are deleted, substituted, inserted, and/or added in the amino acid sequence of SEQ ID NO: 2,
(H3-A) the following amino acid sequence (H3-A1), (H3-A2), or (H3-A3),
(H3-A1) an amino acid sequence of SEQ ID NO: 3,
(H3-A2) an amino acid sequence having 80% or more identity with the amino acid sequence of SEQ ID NO: 3,
(H3-A3) an amino acid sequence in which one or several amino acids are deleted, substituted, inserted, and/or added in the amino acid sequence of SEQ ID NO: 3,
(2) a light chain variable region including a light chain complementarity determining region (CDRL) 1, CDRL2, and CDRL3,
CDRL1 being a polypeptide containing the following amino acid sequence (L1-A),
CDRL2 being a polypeptide containing the following amino acid sequence (L2-A), and
CDRL3 being a polypeptide containing the following amino acid sequence (L3-A),
(L1-A) the following amino acid sequence (L1-A1), (L1-A2), or (L1-A3),
(L1-A1) an amino acid sequence of SEQ ID NO: 4,
(L1-A2) an amino acid sequence having 80% or more identity with the amino acid sequence of SEQ ID NO: 4,
(L1-A3) an amino acid sequence in which one or several amino acids are deleted, substituted, inserted, and/or added in the amino acid sequence of SEQ ID NO: 4,
(L2-A) the following amino acid sequence (L2-A1), (L2-A2), or (L2-A3),
(L2-A1) an amino acid sequence of SEQ ID NO: 5,
(L2-A2) an amino acid sequence having 80% or more identity with the amino acid sequence of SEQ ID NO: 5,
(L2-A3) an amino acid sequence in which one or several amino acids are deleted, substituted, inserted, and/or added in the amino acid sequence of SEQ ID NO: 5,
(L3-A) the following amino acid sequence (L3-A1), (L3-A2), or (L3-A3),
(L3-A1) an amino acid sequence of SEQ ID NO: 6,
(L3-A2) an amino acid sequence having 80% or more identity with the amino acid sequence of SEQ ID NO: 6,
(L3-A3) an amino acid sequence in which one or several amino acids are deleted, substituted, inserted, and/or added in the amino acid sequence of SEQ ID NO: 6,
(B) an antibody against the chikungunya virus, including the following heavy chain variable region (3) and the light chain variable region (2),
(3) a heavy chain variable region including a heavy chain complementarity determining region (CDRH) 1, CDRH2, and CDRH3,
CDRH1 being a polypeptide containing the following amino acid sequence (H1-B),
CDRH2 being a polypeptide containing the following amino acid sequence (H2-B), and
CDRH3 being a polypeptide containing the following amino acid sequence (H3-B),
(H1-B) the following amino acid sequence (H1-B1), (H1-B2), or (H1-B3),
(H1-B1) an amino acid sequence of SEQ ID NO: 7,
(H1-B2) an amino acid sequence having 80% or more identity with the amino acid sequence of SEQ ID NO: 7,
(H1-B3) an amino acid sequence in which one or several amino acids are deleted, substituted, inserted, and/or added in the amino acid sequence of SEQ ID NO: 7,
(H2-B) the following amino acid sequence (H2-B1), (H2-B2), or (H2-B3),
(H2-B1) an amino acid sequence of SEQ ID NO: 8,
(H2-B2) an amino acid sequence having 80% or more identity with the amino acid sequence of SEQ ID NO: 8,
(H2-B3) an amino acid sequence in which one or several amino acids are deleted, substituted, inserted, and/or added in the amino acid sequence of SEQ ID NO: 8,
(H3-B) the following amino acid sequence (H3-B1), (H3-B2), or (H3-B3),
(H3-B1) an amino acid sequence of SEQ ID NO: 9,
(H3-B2) an amino acid sequence having 80% or more identity with the amino acid sequence of SEQ ID NO: 9,
(H3-B3) an amino acid sequence in which one or several amino acids are deleted, substituted, inserted, and/or added in the amino acid sequence of SEQ ID NO: 9,
(C) an antibody against the chikungunya virus, including the following heavy chain variable region (4) and the light chain variable region (2),
(4) a heavy chain variable region including CDRH1, CDRH2, and CDRH3,
CDRH1 being a polypeptide containing the following amino acid sequence (H1-C),
CDRH2 being a polypeptide containing the following amino acid sequence (H2-C), and
CDRH3 being a polypeptide containing the following amino acid sequence (H3-C),
(H1-C) the following amino acid sequence (H1-C1), (H1-C2), or (H1-C3),
(H1-C1) an amino acid sequence of SEQ ID NO: 10,
(H1-C2) an amino acid sequence having 80% or more identity with the amino acid sequence of SEQ ID NO: 10,
(H1-C3) an amino acid sequence in which one or several amino acids are deleted, substituted, inserted, and/or added in the amino acid sequence of SEQ ID NO: 10,
(H2-C) the following amino acid sequence (H2-C1), (H2-C2), or (H2-C3),
(H2-C1) an amino acid sequence of SEQ ID NO: 11,
(H2-C2) an amino acid sequence having 80% or more identity with the amino acid sequence of SEQ ID NO: 11,
(H2-C3) an amino acid sequence in which one or several amino acids are deleted, substituted, inserted, and/or added in the amino acid sequence of SEQ ID NO: 11,
(H3-C) the following amino acid sequence (H3-C1), (H3-C2), or (H3-C3),
(H3-C1) an amino acid sequence of SEQ ID NO: 12,
(H3-C2) an amino acid sequence having 80% or more identity with the amino acid sequence of SEQ ID NO: 12,
(H3-C3) an amino acid sequence in which one or several amino acids are deleted, substituted, inserted, and/or added in the amino acid sequence of SEQ ID NO: 12.

2. An immunochromatographic analysis device for detecting chikungunya virus, comprising a sample loading part, a labeling substance retaining part, a chromatographic medium part having a detection part, and an absorption part, wherein
the labeling substance retaining part contains the following antibody (A) or an antigen-binding fragment thereof, and
the detection part contains the following antibody (B) or an antigen-binding fragment thereof, and the following antibody (C) or an antigen-binding fragment thereof,
(A) an antibody against the chikungunya virus, including the following heavy chain variable region (1) and the following light chain variable region (2),
(1) a heavy chain variable region including a polypeptide composed of the following amino acid sequence (H-A),
(H-A) the following amino acid sequence (H-A1), (H-A2), or (H-A3),
(H-A1) an amino acid sequence of SEQ ID NO: 13,
(H-A2) an amino acid sequence having 80% or more identity with the amino acid sequence of SEQ ID NO: 13,
(H-A3) an amino acid sequence in which one or several amino acids are deleted, substituted, inserted, and/or added in the amino acid sequence of SEQ ID NO: 13,
(2) a light chain variable region including a polypeptide composed of the following amino acid sequence (L-A),
(L-A) the following amino acid sequence (L-A1), (L-A2), or (L-A3),
(L-A1) an amino acid sequence of SEQ ID NO: 14,
(L-A2) an amino acid sequence having 80% or more identity with the amino acid sequence of SEQ ID NO: 14,
(L-A3) an amino acid sequence in which one or several amino acids are deleted, substituted, inserted, and/or added in the amino acid sequence of SEQ ID NO: 14,
(B) an antibody against the chikungunya virus, including the following heavy chain variable region (3) and the light chain variable region (2),
(3) a heavy chain variable region including a polypeptide composed of the following amino acid sequence (H-B),
(H-B) the following amino acid sequence (H-B1), (H-B2), or (H-B3),
(H-B1) an amino acid sequence of SEQ ID NO: 15,
(H-B2) an amino acid sequence having 80% or more identity with the amino acid sequence of SEQ ID NO: 15,
(H-B3) an amino acid sequence in which one or several amino acids are deleted, substituted, inserted, and/or added in the amino acid sequence of SEQ ID NO: 15,
(C) an antibody against the chikungunya virus, including the following heavy chain variable region (4) and the light chain variable region (2),
(4) a heavy chain variable region including a polypeptide composed of the following amino acid sequence (H-C),
(H-C) the following amino acid sequence (H-C1), (H-C2), or (H-C3),
(H-C1) an amino acid sequence of SEQ ID NO: 16,
(H-C2) an amino acid sequence having 80% or more identity with the amino acid sequence of SEQ ID NO: 16,
(H-C3) an amino acid sequence in which one or several amino acids are deleted, substituted, inserted, and/or added in the amino acid sequence of SEQ ID NO: 16.

3. An immunochromatographic analysis device for detecting chikungunya virus, comprising a sample loading part, a labeling substance retaining part, a chromatographic medium part having a detection part, and an absorption part, wherein
the labeling substance retaining part contains the following antibody (A) or an antigen-binding fragment thereof, and
the detection part contains the following antibody (B) or an antigen-binding fragment thereof, and the following antibody (C) or an antigen-binding fragment thereof,
(A) an antibody against the chikungunya virus, including the following heavy chain (1) and the following light chain (2),
(1) a heavy chain including a polypeptide composed of the following amino acid sequence (HA),
(HA) the following amino acid sequence (HA1), (HA2), or (HA3),
(HA1) an amino acid sequence of SEQ ID NO: 17,
(HA2) an amino acid sequence having 80% or more identity with the amino acid sequence of SEQ ID NO: 17,
(HA3) an amino acid sequence in which one or several amino acids are deleted, substituted, inserted, and/or added in the amino acid sequence of SEQ ID NO: 17,
(2) a light chain including a polypeptide composed of the following amino acid sequence (LA),
(LA) the following amino acid sequence (LA1), (LA2), or (LA3),
(LA1) an amino acid sequence of SEQ ID NO: 18,
(LA2) an amino acid sequence having 80% or more identity with the amino acid sequence of SEQ ID NO: 18,
(LA3) an amino acid sequence in which one or several amino acids are deleted, substituted, inserted, and/or added in the amino acid sequence of SEQ ID NO: 18,
(B) an antibody against the chikungunya virus, including the following heavy chain (3) and the light chain (2),
(3) a heavy chain including a polypeptide composed of the following amino acid sequence (HB),
(HB) the following amino acid sequence (HB1), (HB2), or (HB3),
(HB1) an amino acid sequence of SEQ ID NO: 19,
(HB2) an amino acid sequence having 80% or more identity with the amino acid sequence of SEQ ID NO: 19,
(HB3) an amino acid sequence in which one or several amino acids are deleted, substituted, inserted, and/or added in the amino acid sequence of SEQ ID NO: 19,
(C) an antibody against the chikungunya virus, including the following heavy chain (4) and the light chain (2),
(4) a heavy chain including a polypeptide composed of the following amino acid sequence (HC),
(HC) the following amino acid sequence (HC1), (HC2), or (HC3),
(HC1) an amino acid sequence of SEQ ID NO: 20,
(HC2) an amino acid sequence having 80% or more identity with the amino acid sequence of SEQ ID NO: 20,
(HC3) an amino acid sequence in which one or several amino acids are deleted, substituted, inserted, and/or added in the amino acid sequence of SEQ ID NO: 20.

4. The immunochromatographic analysis device according to claim 1, for detecting a chikungunya virus envelope glycoprotein of the chikungunya virus.

5. The immunochromatographic analysis device according to claim 4, wherein the chikungunya virus envelope glycoprotein is chikungunya virus E1 protein.

6. The immunochromatographic analysis device according to claim 1, wherein the antibody (A) or an antigen-binding fragment thereof, the antibody (B) or an antigen-binding fragment thereof, and the antibody (C) or an antigen-binding fragment thereof specifically bind to chikungunya virus having at least any one genotype of ECSA genotype, Asian genotype, and WA genotype.

7. The immunochromatographic analysis device according to claim 1, wherein
the labeling substance retaining part further contains the following antibody (D) or an antigen-binding fragment thereof, and
the detection part further contains the following antibody (E) or an antigen-binding fragment thereof,
(D) an antibody against the chikungunya virus, including the following heavy chain variable region (1) and the following light chain variable region (2),
(1) a heavy chain variable region including a heavy chain complementarity determining region (CDRH) 1, CDRH2, and CDRH3,
CDRH1 being a polypeptide containing the following amino acid sequence (H1-D),
CDRH2 being a polypeptide containing the following amino acid sequence (H2-D), and
CDRH3 being a polypeptide containing the following amino acid sequence (H3-D),
(H1-D) the following amino acid sequence (H1-D1), (H1-D2), or (H1-D3),
(H1-D1) an amino acid sequence of SEQ ID NO: 21,
(H1-D2) an amino acid sequence having 80% or more identity with the amino acid sequence of SEQ ID NO: 21,
(H1-D3) an amino acid sequence in which one or several amino acids are deleted, substituted, inserted, and/or added in the amino acid sequence of SEQ ID NO: 21,
(H2-D) the following amino acid sequence (H2-D1), (H2-D2), or (H2-D3),
(H2-D1) an amino acid sequence of SEQ ID NO: 22,
(H2-D2) an amino acid sequence having 80% or more identity with the amino acid sequence of SEQ ID NO: 22,
(H2-D3) an amino acid sequence in which one or several amino acids are deleted, substituted, inserted, and/or added in the amino acid sequence of SEQ ID NO: 22,
(H3-D) the following amino acid sequence (H3-D1), (H3-D2), or (H3-D3),
(H3-D1) an amino acid sequence of SEQ ID NO: 23,
(H3-D2) an amino acid sequence having 80% or more identity with the amino acid sequence of SEQ ID NO: 23,
(H3-D3) an amino acid sequence in which one or several amino acids are deleted, substituted, inserted, and/or added in the amino acid sequence of SEQ ID NO: 23,
(2) a light chain variable region including a light chain complementarity determining region (CDRL) 1, CDRL2, and CDRL3,
CDRL1 being a polypeptide containing the following amino acid sequence (L1-A),
CDRL2 being a polypeptide containing the following amino acid sequence (L2-A), and
CDRL3 being a polypeptide containing the following amino acid sequence (L3-A),
(L1-A) the following amino acid sequence (L1-A1), (L1-A2), or (L1-A3),
(L1-A1) an amino acid sequence of SEQ ID NO: 4,
(L1-A2) an amino acid sequence having 80% or more identity with the amino acid sequence of SEQ ID NO: 4,
(L1-A3) an amino acid sequence in which one or several amino acids are deleted, substituted, inserted, and/or added in the amino acid sequence of SEQ ID NO: 4,
(L2-A) the following amino acid sequence (L2-A1), (L2-A2), or (L2-A3),
(L2-A1) an amino acid sequence of SEQ ID NO: 5,
(L2-A2) an amino acid sequence having 80% or more identity with the amino acid sequence of SEQ ID NO: 5,
(L2-A3) an amino acid sequence in which one or several amino acids are deleted, substituted, inserted, and/or added in the amino acid sequence of SEQ ID NO: 5,
(L3-A) the following amino acid sequence (L3-A1), (L3-A2), or (L3-A3),
(L3-A1) an amino acid sequence of SEQ ID NO: 6,
(L3-A2) an amino acid sequence having 80% or more identity with the amino acid sequence of SEQ ID NO: 6,
(L3-A3) an amino acid sequence in which one or several amino acids are deleted, substituted, inserted, and/or added in the amino acid sequence of SEQ ID NO: 6,
(E) an antibody against the chikungunya virus, including the following heavy chain variable region (3) and the light chain variable region (2),
(3) a heavy chain variable region including a heavy chain complementarity determining region (CDRH) 1, CDRH2, and CDRH3,
CDRH1 being a polypeptide containing the following amino acid sequence (H1-E),
CDRH2 being a polypeptide containing the following amino acid sequence (H2-E), and
CDRH3 being a polypeptide containing the following amino acid sequence (H3-E),
(H1-E) the following amino acid sequence (H1-E1), (H1-E2), or (H1-E3),
(H1-E1) an amino acid sequence of SEQ ID NO: 24,
(H1-E2) an amino acid sequence having 80% or more identity with the amino acid sequence of SEQ ID NO: 24,
(H1-E3) an amino acid sequence in which one or several amino acids are deleted, substituted, inserted, and/or added in the amino acid sequence of SEQ ID NO: 24,
(H2-E) the following amino acid sequence (H2-E1), (H2-E2), or (H2-E3),
(H2-E1) an amino acid sequence of SEQ ID NO: 25,
(H2-E2) an amino acid sequence having 80% or more identity with the amino acid sequence of SEQ ID NO: 25,
(H2-E3) an amino acid sequence in which one or several amino acids are deleted, substituted, inserted, and/or added in the amino acid sequence of SEQ ID NO: 25,
(H3-E) the following amino acid sequence (H3-E1), (H3-E2), or (H3-E3),
(H3-E1) an amino acid sequence of SEQ ID NO: 26,
(H3-E2) an amino acid sequence having 80% or more identity with the amino acid sequence of SEQ ID NO: 26,
(H3-E3) an amino acid sequence in which one or several amino acids are deleted, substituted, inserted, and/or added in the amino acid sequence of SEQ ID NO: 26.

8. The immunochromatographic analysis device according to claim 1, wherein
the labeling substance retaining part further contains the following antibody (D) or an antigen-binding fragment thereof, and
the detection part further contains the following antibody (E) or an antigen-binding fragment thereof,
(D) an antibody against the chikungunya virus, including the following heavy chain variable region (1) and the following light chain variable region (2),
(1) a heavy chain variable region including a polypeptide composed of the following amino acid sequence (H-D),
(H-D) the following amino acid sequence (H-D1), (H-D2), or (H-D3),
(H-D1) an amino acid sequence of SEQ ID NO: 27,
(H-D2) an amino acid sequence having 80% or more identity with the amino acid sequence of SEQ ID NO: 27,
(H-D3) an amino acid sequence in which one or several amino acids are deleted, substituted, inserted, and/or added in the amino acid sequence of SEQ ID NO: 27,
(2) a light chain variable region including a polypeptide composed of the following amino acid sequence (L-A),
(L-A) the following amino acid sequence (L-A1), (L-A2), or (L-A3),
(L-A1) an amino acid sequence of SEQ ID NO: 14,
(L-A2) an amino acid sequence having 80% or more identity with the amino acid sequence of SEQ ID NO: 14,
(L-A3) an amino acid sequence in which one or several amino acids are deleted, substituted, inserted, and/or added in the amino acid sequence of SEQ ID NO: 14,
(E) an antibody against the chikungunya virus, including the following heavy chain variable region (3) and the light chain variable region (2),
(3) a heavy chain variable region including a polypeptide composed of the following amino acid sequence (H-E),
(H-E) the following amino acid sequence (H-E1), (H-E2), or (H-E3),
(H-E1) an amino acid sequence of SEQ ID NO: 28,
(H-E2) an amino acid sequence having 80% or more identity with the amino acid sequence of SEQ ID NO: 28,
(H-E3) an amino acid sequence in which one or several amino acids are deleted, substituted, inserted, and/or added in the amino acid sequence of SEQ ID NO: 28.

9. The immunochromatographic analysis device according to claim 1, wherein
the labeling substance retaining part further contains the following antibody (D) or an antigen-binding fragment thereof, and
the detection part further contains the following antibody (E) or an antigen-binding fragment thereof,
(D) an antibody against the chikungunya virus, including the following heavy chain (1) and the following light chain (2),
(1) a heavy chain including a polypeptide composed of the following amino acid sequence (HD),
(HD) the following amino acid sequence (HD1), (HD2), or (HD3),
(HD1) an amino acid sequence of SEQ ID NO: 29,
(HD2) an amino acid sequence having 80% or more identity with the amino acid sequence of SEQ ID NO: 29,
(HD3) an amino acid sequence in which one or several amino acids are deleted, substituted, inserted, and/or added in the amino acid sequence of SEQ ID NO: 29,
(2) a light chain including a polypeptide composed of the following amino acid sequence (LA),
(LA) the following amino acid sequence (LA1), (LA2), or (LA3),
(LA1) an amino acid sequence of SEQ ID NO: 18,
(LA2) an amino acid sequence having 80% or more identity with the amino acid sequence of SEQ ID NO: 18,
(LA3) an amino acid sequence in which one or several amino acids are deleted, substituted, inserted, and/or added in the amino acid sequence of SEQ ID NO: 18,
(E) an antibody against the chikungunya virus, including the following heavy chain (3) and the light chain (2),
(3) a heavy chain including a polypeptide composed of the following amino acid sequence (HE),
(HE) the following amino acid sequence (HE1), (HE2), or (HE3),
(HE1) an amino acid sequence of SEQ ID NO: 30,
(HE2) an amino acid sequence having 80% or more identity with the amino acid sequence of SEQ ID NO: 30,
(HE3) an amino acid sequence in which one or several amino acids are deleted, substituted, inserted, and/or added in the amino acid sequence of SEQ ID NO: 30.

10. The immunochromatographic analysis device according to claim 7, wherein the antibody (D) or an antigen-binding fragment thereof, and the antibody (E) or an antigen-binding fragment thereof specifically bind to chikungunya virus having at least any one genotype of ECSA genotype, Asian genotype, and WA genotype.

11. The immunochromatographic analysis device according to claim 7, wherein a content ratio (mass) of the antibody (A) or an antigen-binding fragment thereof to the antibody (D) or an antigen-binding fragment thereof in the labeling substance retaining part is 1:2 to 2:1.

12. The immunochromatographic analysis device according to claim 1, wherein a sample to be loaded in the sample loading part is any one of the whole blood, serum, plasma, semen, and spinal fluid of a chikungunya virus infected individual.

13. An immunochromatographic analysis kit, comprising the immunochromatographic analysis device according to claim 1, and a specimen diluent for diluting and developing a specimen.

14. An immunochromatographic analysis method for detecting chikungunya virus contained in a specimen using the immunochromatographic analysis kit according to claim 13, comprising the following steps (1) to (4):
(1) a step of loading a specimen-containing liquid obtained by diluting a specimen with a specimen diluent in the sample loading part;
(2) a step of allowing the antibody (A) or an antigen-binding fragment thereof held by the labeling substance retaining part to recognize the chikungunya virus;
(3) a step of developing the specimen and the antibody (A) or an antigen-binding fragment thereof in the chromatographic medium part as a mobile phase; and
(4) a step of detecting the chikungunya virus in the developed mobile phase with the antibody (B) or an antigen-binding fragment thereof and the antibody (C) or an antigen-binding fragment thereof contained in the detection part.

15. The immunochromatographic analysis device according to claim 2, for detecting a chikungunya virus envelope glycoprotein of the chikungunya virus.

16. The immunochromatographic analysis device according to claim 3, for detecting a chikungunya virus envelope glycoprotein of the chikungunya virus.

17. The immunochromatographic analysis device according to claim 15, wherein the chikungunya virus envelope glycoprotein is chikungunya virus E1 protein.

18. The immunochromatographic analysis device according to claim 16, wherein the chikungunya virus envelope glycoprotein is chikungunya virus E1 protein.

19. The immunochromatographic analysis device according to claim 2, wherein the antibody (A) or an antigen-binding fragment thereof, the antibody (B) or an antigen-binding fragment thereof, and the antibody (C) or an antigen-binding fragment thereof specifically bind to chikungunya virus having at least any one genotype of ECSA genotype, Asian genotype, and WA genotype.

20. The immunochromatographic analysis device according to claim 3, wherein the antibody (A) or an antigen-binding fragment thereof, the antibody (B) or an antigen-binding fragment thereof, and the antibody (C) or an antigen-binding fragment thereof specifically bind to chikungunya virus having at least any one genotype of ECSA genotype, Asian genotype, and WA genotype.


SEQUENCE LISTING (33 sequences)

SEQ ID NO: 1 - protein, 8 aa, Artificial Sequence
  CDRH1 of 13H11 heavy chain
GYAFSTSW

SEQ ID NO: 2 - protein, 8 aa, Artificial Sequence
  CDRH2 of 13H11 heavy chain
IYPGDGDT

SEQ ID NO: 3 - protein, 9 aa, Artificial Sequence
  CDRH3 of 13H11 heavy chain
SNDGYYVGY

SEQ ID NO: 4 - protein, 6 aa, Artificial Sequence
  CDRL1 of 13H11, 3D11, 15B2, CK-(d) and CK-(e) light chain
ENVVTY

SEQ ID NO: 5 - protein, 3 aa, Artificial Sequence
  CDRL2 of 13H11, 3D11, 15B2, CK-(d) and CK-(e) light chain
GAS

SEQ ID NO: 6 - protein, 9 aa, Artificial Sequence
  CDRL3 of 13H11, 3D11, 15B2, CK-(d) and CK-(e) light chain
GQGYSYPYT

SEQ ID NO: 7 - protein, 8 aa, Artificial Sequence
  CDRH1 of 3D11 heavy chain
GYTFTSYW

SEQ ID NO: 8 - protein, 11 aa, Artificial Sequence
  CDRH2 of 3D11 heavy chain
IYPGDGDTRYT

SEQ ID NO: 9 - protein, 7 aa, Artificial Sequence
  CDRH3 of 3D11 heavy chain
SYDPFDY

SEQ ID NO: 10 - protein, 8 aa, Artificial Sequence
  CDRH1 of 15B2 heavy chain
GYTFTSYY

SEQ ID NO: 11 - protein, 8 aa, Artificial Sequence
  CDRH2 of 15B2 heavy chain
INPSNGGT

SEQ ID NO: 12 - protein, 10 aa, Artificial Sequence
  CDRH3 of 15B2 heavy chain
GYYGNPFFAY

SEQ ID NO: 13 - protein, 118 aa, Artificial Sequence
  Variable region of 13H11 heavy chain
QVQLQQSGPELVKPGASVKISCKASGYAFSTSWMNWVKQRPGQGLEWIGRIYPGDGDTNYNGKFKGKATLTADKSSSTAYMQLSSLTSVDSAVYFCARSNDGYYVGYWGQGTTLTVSS

SEQ ID NO: 14 - protein, 106 aa, Artificial Sequence
  Variable region of 13H11, 3D11, 15B2, CK-(d) and CK-(e) light chain
NIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEI

SEQ ID NO: 15 - protein, 116 aa, Artificial Sequence
  Variable region of 3D11 heavy chain
QVQLQQSGAELARPGASVKLSCKASGYTFTSYWMQWVKQRPGQGLEWIGAIYPGDGDTRYTQKFKGKATLTADKSSSTAYMQLSSLASEDSAVYYCARSYDPFDYWGQGTTLTVSS

SEQ ID NO: 16 - protein, 119 aa, Artificial Sequence
  Variable region of 15B2 heavy chain
QVQLQQSGAELVKPGASVKLSCKASGYTFTSYYMYWVKQRPGQGLEWIGEINPSNGGTNFNEKFKNKATLTVDKSSNTAYMQLNSLTSEDSAVYYCTRGYYGNPFFAYWGQGTLVTVSA

SEQ ID NO: 17 - protein, 442 aa, Artificial Sequence
  13H11 heavy chain
QVQLQQSGPELVKPGASVKISCKASGYAFSTSWMNWVKQRPGQGLEWIGRIYPGDGDTNYNGKFKGKATLTADKSSSTAYMQLSSLTSVDSAVYFCARSNDGYYVGYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 18 - protein, 214 aa, Artificial Sequence
  13H11, 3D11, 15B2, CK-(d) and CK-(e)  light chain
NIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 19 - protein, 446 aa, Artificial Sequence
  3D11 heavy chain
QVQLQQSGAELARPGASVKLSCKASGYTFTSYWMQWVKQRPGQGLEWIGAIYPGDGDTRYTQKFKGKATLTADKSSSTAYMQLSSLASEDSAVYYCARSYDPFDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 20 - protein, 455 aa, Artificial Sequence
  15B2 heavy chain
QVQLQQSGAELVKPGASVKLSCKASGYTFTSYYMYWVKQRPGQGLEWIGEINPSNGGTNFNEKFKNKATLTVDKSSNTAYMQLNSLTSEDSAVYYCTRGYYGNPFFAYWGQGTLVTVSAAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 21 - protein, 8 aa, Artificial Sequence
  CDRH1 of CK-(d) heavy chain
GFTFSTYY

SEQ ID NO: 22 - protein, 8 aa, Artificial Sequence
  CDRH2 of CK-(d) heavy chain
INSNGGST

SEQ ID NO: 23 - protein, 12 aa, Artificial Sequence
  CDRH3 of CK-(d) heavy chain
ARHELVGGWFVY

SEQ ID NO: 24 - protein, 8 aa, Artificial Sequence
  CDRH1 of CK-(e) heavy chain
GYTFTNYW

SEQ ID NO: 25 - protein, 8 aa, Artificial Sequence
  CDRH2 of CK-(e) heavy chain
VYPGGGST

SEQ ID NO: 26 - protein, 13 aa, Artificial Sequence
  CDRH3 of CK-(e) heavy chain
SRVTSTTGWYFDV

SEQ ID NO: 27 - protein, 114 aa, Artificial Sequence
  Variable region of CK-(d) heavy chain
ESGGGLVKLGGSLKLSCAASGFTFSTYYMSWVRQTPEKRLELVAAINSNGGSTYYPDTVKGRFTISRDNAKNTLYLQMSSLKSEDTALYYCARHELVGGWFVYWGQGTLVTVSA

SEQ ID NO: 28 - protein, 120 aa, Artificial Sequence
  Variable region of CK-(e) heavy chain
QVQLQQSGAELVRPGTSVKMSCKAAGYTFTNYWIGWIKQRPGHGLEWIGDVYPGGGSTYYNEKFKAKATLTADTSSSTAYMQLSRLTSEDSAIYYCSRVTSTTGWYFDVWGAGTTVTVSS

SEQ ID NO: 29 - protein, 438 aa, Artificial Sequence
  CK-(d) heavy chain
ESGGGLVKLGGSLKLSCAASGFTFSTYYMSWVRQTPEKRLELVAAINSNGGSTYYPDTVKGRFTISRDNAKNTLYLQMSSLKSEDTALYYCARHELVGGWFVYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 30 - protein, 450 aa, Artificial Sequence
  CK-(e) heavy chain
QVQLQQSGAELVRPGTSVKMSCKAAGYTFTNYWIGWIKQRPGHGLEWIGDVYPGGGSTYYNEKFKAKATLTADTSSSTAYMQLSRLTSEDSAIYYCSRVTSTTGWYFDVWGAGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 31 - protein, 981 aa, Chikungunya virus
MCLLANTTFPCSQPPCTPCCYEKEPEETLRMLEDNVMRPGYYQLLQASLTCSPHRQRRSTKDNFNVYKATRPYLAHCPDCGEGHSCHSPVALERIRNEATDGTLKIQVSLQIGIKTDDSHDWTKLRYMDNHMPADAERAGLFVRTSAPCTITGTMGHFILARCPKGETLTVGFTDSRKISHSCTHPFHHDPPVIGREKFHSRPQHGKELPCSTYVQSTAATTEEIEVHMPPDTPDRTLMSQQSGNVKITVNGQTVRYKCNCGGSNEGLTTTDKVINNCKVDQCHAAVTNHKKWQYNSPLVPRNAELGDRQGKIHIPFPLANVTCRVPKARNPTVTYGKNQVIMLLYPDHPTLLSYRNMGEEPNYQEEWVMHKKEVVLTVPTEGLEVTWGNNEPYKYWPQLSTNGTAHGHPHEIILYYYELYPTMTVVVVSVATFILLSMVGMAAGMCMCARRRCITPYELTPGATVPFLLSLICCIRTAKAATYQEAAIYLWNEQQPLFWLQALIPLAALIVLCNCLRLLPCCCKTLAFLAVMSVGAHTVSAYEHVTVIPNTVGVPYKTLVNRPGYSPMVLEMELLSVTLEPTLSLDYITCEYKTVIPSPYVKCCGTAECKDKNLPDYSCKVFTGVYPFMWGGAYCFCDAENTQLSEAHVEKSESCKTEFASAYRAHTASASAKLRVLYQGNNITVTAYANGDHAVTVKDAKFIVGPMSSAWTPFDNKIVVYKGDVYNMDYPPFGAGRPGQFGDIQSRTPESKDVYANTQLVLQRPAVGTVHVPYSQAPSGFKYWLKERGASLQHTAPFGCQIATNPVRAVNCAVGNMPISIDIPEAAFTRVVDAPSLTDMSCEVPACTHSSDFGGVAIIKYAASKKGKCAVHSMTNAVTIREAEIEVEGNSQLQISFSTALASAEFRVQVCSTQVHCAAECHPPKDHIVNYPASHTTLGVQDISATAMSWVQKITGGVGLVVAVAALILIVVLCVSFSRH

SEQ ID NO: 32 - protein, 981 aa, Chikungunya virus
LCLLANTTFPCSQPPCTPCCYEKEPESTLRMLEDNVMRPGYYQLLKASLTCSPHRQRRSTKDNFNVYKATRPYLAHCPDCGEGHSCHSPIALERIRNEATDGTLKIQVSLQIGIKTDDSHDWTKLRYMDSHTPADAERAGLLVRTSAPCTITGTMGHFILARCPKGETLTVGFTDSRKISHTCTHPFHHEPPVIGRERFHSRPQHGKELPCSTYVQSTAATAEEIEVHMPPDTPDRTLMTQQSGNVKITVNGQTVRYKCNCGGSNEGLTTTDKVINNCKIDQCHAAVTNHKNWQYNSPLVPRNAELGDRKGKIHIPFPLANVTCRVPKARNPTVTYGKNQVTMLLYPDHPTLLSYRNMGQEPNYHEEWVTHKKEVTLTVPTEGLEVTWGNNEPYKYWPQMSTNGTAHGHPHEIILYYYELYPTMTVVIVSVASFVLLSMVGTAVGMCVCARRRCITPYELTPGATVPFLLSLLCCVRTTKAATYYEAAAYLWNEQQPLFWLQALIPLAALIVLCNCLKLLPCCCKTLAFLAVMSIGAHTVSAYEHVTVIPNTVGVPYKTLVNRPGYSPMVLEMELQSVTLEPTLSLDYITCEYKTVIPSPYVKCCGTAECKDKSLPDYSCKVFTGVYPFMWGGAYCFCDAENTQLSEAHVEKSESCKTEFASAYRAHTASASAKLRVLYQGNNITVAAYANGDHAVTVKDAKFVVGPMSSAWTPFDNKIVVYKGDVYNMDYPPFGAGRPGQFGDIQSRTPESKDVYANTQLVLQRPAAGTVHVPYSQAPSGFKYWLKERGASLQHTAPFGCQIATNPVRAVNCAVGNIPISIDIPDAAFTRVVDAPSVTDMSCEVPACTHSSDFGGVAIIKYTASKKGKCAVHSMTNAVTIREADVEVEGNSQLQISFSTALASAEFRVQVCSTQVHCAAACHPPKDHIVNYPASHTTLGVQDISTTAMSWVQKITGGVGLIVAVAALILIVVLCVSFSRH

SEQ ID NO: 33 - protein, 981 aa, Chikungunya virus
MCLLANTTFPCSQPPCTPCCYEKEPEKTLRMLEDNVMSPGYYQLLQASLTCSPRRQRRSIKDNFNVYKATRPYLAHCPDCGEGHSCHSPVALERIRNEATDGTLKIQVSLQIGIKTDDSHDWTKLRYMDNHMPADAERAGLFVRTSAPCTITGTMGHFILARCPKGETLTVGFTDGRKISHSCTHPFHHDPPVIGREKFHSRPQHGRELPCSTYAQSTAATAEEIEVHMPPDTPDRTLMSQQSGNVKITVNSQTVRYKCNCGDSNEGLTTTDKVINNCKVDQCHAAVTNHKKWQYNSPLVPRNAELGDRKGKVHIPFPLANVTCRVPKARNPTVTYGKNQVIMLLYPDHPTLLSYRNMGEEPNYQEEWVTHKKEIRLTVPTEGLEVTWGNNEPYKYWPQLSTNGTAHGHPHEIILYYYELYPTMTVVVVSVASFVLLSMVGVAVGMCMCARRRCITPYELTPGATVPFLLSLICCIRTAKAATYQEAAVYLWNEQQPLFWLQALIPLAALIVLCNCLRLLPCCCKTLTFLAVLSVGAHTVSAYEHVTVIPNTVGVPYKTLVNRPGYSPMVLEMELLSVTLEPTLSLDYITCEYKTVIPSPYVKCCGTAECKDKSLPDYSCKVFTGVYPFMWGGAYCFCDTENTQLSEAHVEKSESCKTEFASAYRAHTASASAKLRVLYQGNNVTVSAYANGDHAVTVKDAKFIVGPMSSAWTPFDNKIVVYKGDVYNMDYPPFGAGRPGQFGDIQSRTPESEDVYANTQLVLQRPSAGTVHVPYSQAPSGFKYWLKERGASLQHTAPFGCQIATNPVRAMNCAVGNMPISIDIPDAAFTRVVDAPSLTDMSCEVSACTHSSDFGGVAIIKYAASKKGKCAVHSMTNAVTIREAEIEVEGNSQLQISFSTALASAEFRVQVCSTQVHCAAECHPPKDHIVNYPASHTTLGVQDISATAMSWVQKITGGVGLVVAVAALILIVVLCVSFSRH